SEQUENCE LISTING (70 sequences)

SEQ ID NO: 1 - protein, 119 aa, Artificial Sequence
  variable region of heavy chain
EVHLQQSGPEVGRPGSSVKISCKASGYTFTDYIIHWVKQSPGQGLEWMGWIDPDYGNTDYADKFKKKATLTADASSNTAFIHLSRLTSEDTATYFCARDDGSPLVMDAWGQGTSVTVSS

SEQ ID NO: 2 - protein, 10 aa, Artificial Sequence
  HCDR1
GYTFTDYIIH

SEQ ID NO: 3 - protein, 17 aa, Artificial Sequence
  HCDR2
WIDPDYGNTDYADKFKK

SEQ ID NO: 4 - protein, 10 aa, Artificial Sequence
  HCDR3
DDGSPLVMDA

SEQ ID NO: 5 - protein, 106 aa, Artificial Sequence
  variable region of light chain
NIQLTQSPSLLSASVGDRVTLSCNGSQNIDNYLAWFQQKLGEAPKLLIYSTYSLQTGIPSRFSGSRSGTDYTLTISSLQPEDVATYFCFQYNNGYMFGAGTKLELK

SEQ ID NO: 6 - protein, 11 aa, Artificial Sequence
  LCDR1
NGSQNIDNYLA

SEQ ID NO: 7 - protein, 7 aa, Artificial Sequence
  LCDR2
STYSLQT

SEQ ID NO: 8 - protein, 8 aa, Artificial Sequence
  LCDR3
FQYNNGYM

SEQ ID NO: 9 - protein, 118 aa, Artificial Sequence
  variable region of heavy chain
EVQLVETGGGLVQPGKSLKLTCATSGFTFSNAWMGWVRQSPEKQLEWVAQIKDKSNNYPTYYAESVKGRFTISRDDSESSVYLQMNSLKEEDTAIYYCTGYGDFFDYWGQGVVVTVSS

SEQ ID NO: 10 - protein, 10 aa, Artificial Sequence
  HCDR1
GFTFSNAWMG

SEQ ID NO: 11 - protein, 19 aa, Artificial Sequence
  HCDR2
QIKDKSNNYPTYYAESVKG

SEQ ID NO: 12 - protein, 7 aa, Artificial Sequence
  HCDR3
YGDFFDY

SEQ ID NO: 13 - protein, 107 aa, Artificial Sequence
  variable region of light chain
DIQMTQSPASLSASLGETVTIECRASEDIYNGLAWYQQKPGNSPRLLIFNANSLHTGVPSRFSGSGSGTQYSLKINSLQSEDVASYFCQQYYDHPLTFGSGIKLEIK

SEQ ID NO: 14 - protein, 11 aa, Artificial Sequence
  LCDR1
RASEDIYNGLA

SEQ ID NO: 15 - protein, 7 aa, Artificial Sequence
  LCDR2
NANSLHT

SEQ ID NO: 16 - protein, 9 aa, Artificial Sequence
  LCDR3
QQYYDHPLT

SEQ ID NO: 17 - protein, 118 aa, Artificial Sequence
  variable region of heavy chain
EVQLQQSGPELRRPGSSVKLSCKASGYSITDYLLHWVKHSPEHGLEWIGWIDPEDGKTKYGQKFQSKATLTADTSSNAAYMQLSSLTSEDSATYFCAANSGTGTMDAWGQGASVTVSS

SEQ ID NO: 18 - protein, 10 aa, Artificial Sequence
  HCDR1
GYSITDYLLH

SEQ ID NO: 19 - protein, 17 aa, Artificial Sequence
  HCDR2
WIDPEDGKTKYGQKFQS

SEQ ID NO: 20 - protein, 9 aa, Artificial Sequence
  HCDR3
NSGTGTMDA

SEQ ID NO: 21 - protein, 107 aa, Artificial Sequence
  variable region of light chain
DIVMTQSPTSMSISVGDRVTLNCKASQNVGSNVDWYQQKTGQSPKVLLYKASIRYTGVPDRFTGSGSGTEFTFTITNMQAEDLAVYFCMQSKSYPITFGSGTKLEIK

SEQ ID NO: 22 - protein, 11 aa, Artificial Sequence
  LCDR1
KASQNVGSNVD

SEQ ID NO: 23 - protein, 7 aa, Artificial Sequence
  LCDR2
KASIRYT

SEQ ID NO: 24 - protein, 9 aa, Artificial Sequence
  LCDR3
MQSKSYPIT

SEQ ID NO: 25 - protein, 119 aa, Artificial Sequence
  variable region of heavy chain
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDDGSPLVMDAWGQGTLVTVSS

SEQ ID NO: 26 - protein, 106 aa, Artificial Sequence
  variable region of light chain
DIQMTQSPSSLSASVGDRVTITCQGSQNIDNYLAWFQQKPGKAPKLLIYSTYSLQTGVPSRFSGSRSGTDFTLTISSLQPEDFATYFCFQYNNGYMFGGGTKLELK

SEQ ID NO: 27 - protein, 11 aa, Artificial Sequence
  LCDR1
RASEDIYQGLA

SEQ ID NO: 28 - protein, 11 aa, Artificial Sequence
  LCDR1
QGSQNIDNYLA

SEQ ID NO: 29 - protein, 119 aa, Artificial Sequence; identical to SEQ ID NO: 25
  variable region of heavy chain

SEQ ID NO: 30 - protein, 106 aa, Artificial Sequence
  variable region of light chain
DIQMTQSPSSLSASVGDRVTITCQGSQNIDNYLAWFQQKPGKAPKLLIYSTYSLQTGVPSRFSGSRSGTDFTLTISSLQPEDFATYFCFQYNQGYMFGGGTKLELK

SEQ ID NO: 31 - protein, 119 aa, Artificial Sequence; identical to SEQ ID NO: 25
  variable region of heavy chain

SEQ ID NO: 32 - protein, 106 aa, Artificial Sequence
  variable region of light chain
DIQMTQSPSSLSASVGDRVTITCNGAQNIDNYLAWFQQKPGKAPKLLIYSTYSLQTGVPSRFSGSRSGTDFTLTISSLQPEDFATYFCFQYNNGYMFGGGTKLELK

SEQ ID NO: 33 - protein, 11 aa, Artificial Sequence
  LCDR1
NGAQNIDNYLA

SEQ ID NO: 34 - protein, 119 aa, Artificial Sequence
  variable region of heavy chain
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDDASPLVMDAWGQGTLVTVSS

SEQ ID NO: 35 - protein, 106 aa, Artificial Sequence
  variable region of light chain
DIQMTQSPSSLSASVGDRVTITCNGSQNIDNYLAWFQQKPGKAPKLLIYSTYSLQTGVPSRFSGSRSGTDFTLTISSLQPEDFATYFCFQYNNGYMFGGGTKLELK

SEQ ID NO: 36 - protein, 10 aa, Artificial Sequence
  HCDR3
DDASPLVMDA

SEQ ID NO: 37 - protein, 119 aa, Artificial Sequence; identical to SEQ ID NO: 34
  variable region of heavy chain

SEQ ID NO: 38 - protein, 106 aa, Artificial Sequence; identical to SEQ ID NO: 26
  variable region of light chain

SEQ ID NO: 39 - protein, 119 aa, Artificial Sequence; identical to SEQ ID NO: 34
  variable region of heavy chain

SEQ ID NO: 40 - protein, 106 aa, Artificial Sequence; identical to SEQ ID NO: 30
  variable region of light chain

SEQ ID NO: 41 - protein, 119 aa, Artificial Sequence
  variable region of heavy chain
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDEGSPLVMDAWGQGTLVTVSS

SEQ ID NO: 42 - protein, 106 aa, Artificial Sequence; identical to SEQ ID NO: 35
  variable region of light chain

SEQ ID NO: 43 - protein, 119 aa, Artificial Sequence; identical to SEQ ID NO: 41
  variable region of heavy chain

SEQ ID NO: 44 - protein, 106 aa, Artificial Sequence; identical to SEQ ID NO: 26
  variable region of light chain

SEQ ID NO: 45 - protein, 10 aa, Artificial Sequence
  HCDR3
DEGSPLVMDA

SEQ ID NO: 46 - protein, 119 aa, Artificial Sequence; identical to SEQ ID NO: 41
  variable region of heavy chain

SEQ ID NO: 47 - protein, 122 aa, Artificial Sequence
  variable region of light chain
DIVMTQSPATLSVSPGERATLSCKASQNVGSNVDWYQQKPGQSPRVLIYWYQQKPGQSPRVLIYKASIRYTGIPARFSGSGSGTEFTLTISILQSEDFAVYFCMQSKSYPITFGQGTKLEIK

SEQ ID NO: 48 - protein, 8 aa, Artificial Sequence
  LCDR3
FQYNQGYM

SEQ ID NO: 49 - protein, 118 aa, Artificial Sequence
  variable region of heavy chain
EVQLVESGGGLVKPGGSLRLSCAASGFTFSNAWMGWVRQAPEKGLEWVGQIKDKSNNYPTYYAESVKGRFTISRDDSKNTLYLQMNSLKTEDTAVYYCTTYGDFFDYWGQGTLVTVSS

SEQ ID NO: 50 - protein, 107 aa, Artificial Sequence
  variable region of light chain
DIQMTQSPSSLSASVGDRVTITCRASEDIYNALAWYQQKPGKVPKLLLYNANSLHTGVPSRFSGSGSGTDYTLTISSLQPEDFATYFCQQYYDHPLTFGQGTKLEIK

SEQ ID NO: 51 - protein, 118 aa, Artificial Sequence; identical to SEQ ID NO: 49
  variable region of heavy chain

SEQ ID NO: 52 - protein, 107 aa, Artificial Sequence
  variable region of light chain
DIQMTQSPSSLSASVGDRVTITCRASEDIYQGLAWYQQKPGKVPKLLLYNANSLHTGVPSRFSGSGSGTDYTLTISSLQPEDFATYFCQQYYDHPLTFGQGTKLEIK

SEQ ID NO: 53 - protein, 118 aa, Artificial Sequence
  variable region of heavy chain
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAANSGTGTMDAWGQGTLVTVSS

SEQ ID NO: 54 - protein, 107 aa, Artificial Sequence
  variable region of light chain
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIK

SEQ ID NO: 55 - protein, 17 aa, Artificial Sequence
  HCDR2
WIDPEEGKTKYGQKFQS

SEQ ID NO: 56 - protein, 118 aa, Artificial Sequence
  variable region of heavy chain
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSS

SEQ ID NO: 57 - protein, 107 aa, Artificial Sequence; identical to SEQ ID NO: 54
  variable region of light chain

SEQ ID NO: 58 - protein, 17 aa, Artificial Sequence
  HCDR2
WIDPEDAKTKYGQKFQS

SEQ ID NO: 59 - protein, 118 aa, Artificial Sequence
  variable region of heavy chain
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAAQSGTGTMDAWGQGTLVTVSS

SEQ ID NO: 60 - protein, 107 aa, Artificial Sequence; identical to SEQ ID NO: 54
  variable region of light chain

SEQ ID NO: 61 - protein, 9 aa, Artificial Sequence
  HCDR3
QSGTGTMDA

SEQ ID NO: 62 - protein, 118 aa, Artificial Sequence
  variable region of heavy chain
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAANSATGTMDAWGQGTLVTVSS

SEQ ID NO: 63 - protein, 107 aa, Artificial Sequence; identical to SEQ ID NO: 54
  variable region of light chain

SEQ ID NO: 64 - protein, 9 aa, Artificial Sequence
  HCDR3
NSATGTMDA

SEQ ID NO: 65 - protein, 118 aa, Artificial Sequence
  variable region of heavy chain
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAAQSGTGTMDAWGQGTLVTVSS

SEQ ID NO: 66 - protein, 107 aa, Artificial Sequence; identical to SEQ ID NO: 54
  variable region of light chain

SEQ ID NO: 67 - protein, 118 aa, Artificial Sequence
  variable region of heavy chain
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSATGTMDAWGQGTLVTVSS

SEQ ID NO: 68 - protein, 122 aa, Artificial Sequence; identical to SEQ ID NO: 47
  variable region of light chain

SEQ ID NO: 69 - protein, 107 aa, Artificial Sequence [sequence omitted]

SEQ ID NO: 70 - protein, 330 aa, Artificial Sequence [sequence omitted]

(19) United States
(12) Patent Application Publication
(10) Pub. No.: US 2025/0320303 A1
(43) Pub. Date: Oct. 16, 2025

(54) ANTI-TNFR2 ANTIBODY AND PREPARATION METHOD AND APPLICATION THEREOF

(71) Applicant: SHENGHE (CHINA) BIOPHARMACEUTICAL CO., LTD., Nanjing, Jiangsu (CN)

(72) Inventors: Xiaoling JIANG, Nanjing (CN); Liusong YIN, Nanjing (CN); Chongbing WU, Nanjing (CN); Jinhua ZHOU, Nanjing (CN)

(73) Assignee: SHENGHE (CHINA) BIOPHARMACEUTICAL CO., LTD., Nanjing, Jiangsu (CN)

(21) Appl. No.: 18/569,312

(22) PCT Filed: Jun. 14, 2022
(86) PCT No.: PCT/CN2022/098506

§ 371 Date: Dec. 12, 2023

(30) Foreign Application Priority Data
Jun. 22, 2021 (WO) PCT/CN2021/101417

(51) Int. Cl. C07K 16/28; A61K 39/00; A61K 45/06; A61K 47/68; A61P 35/00

(52) U.S. Cl. CPC C07K 16/2878; A61K 45/06; A61K 47/6849; A61P 35/00; A61K 2039/505; C07K 2317/24; C07K 2317/565; C07K 2317/73; C07K 2317/75; C07K 2317/92

(57) ABSTRACT

An antibody or antigen-binding fragment thereof that binds specifically to TNFR2 with high affinity. A nucleic acid molecule encoding the antibody or antigen-binding fragment thereof, an expression vector and a host cell for expressing the antibody or antigen-binding fragment thereof, and a method for producing the antibody or antigen-binding fragment thereof are also provided. Also included are an antibody-drug conjugate and a drug composition including the antibody or antigen-binding fragment thereof, and a combination therapy with other therapeutic agents, as well as the preparation of a drug for the treatment of a variety of diseases using the antibody or antigen-binding fragment thereof.

### TECHNICAL FIELD

The present invention belongs to the field of biomedicine, and specifically relates to an anti-TNFR2 antibody and preparation method and use thereof.

### BACKGROUND

Recent studies have shown that enhancing the body's own ability to fight disease by modulating the immune response is an effective alternative and/or complement to traditional therapeutic methods. For example, studies have shown that enhancing activity against T lymphocytes to target and treat various diseases (e.g., cancer or autoimmune diseases) is therapeutically effective. The immune response against disease can be enhanced by inhibiting the ability of Treg to suppress T lymphocyte activity or by enhancing the ability of effector T cells to suppress tumors.

Tumor necrosis factor receptor 2 (TNFR2), also known as TNFRSFlB and CD120b, is a co-stimulatory member of the tumor necrosis factor receptor superfamily (TNFRSF), which includes proteins such as GITR, OX40, CD27, CD40, and 4-IBB (CD137). TNFR2 is a cell surface receptor expressed on T cells and has been shown to enhance effector T cell activation and reduce Treg-mediated inhibition. By regulating TRAF2/3 and NF-kB signaling, TNFR2 can mediate transcription of genes that promote cell survival and proliferation. TNFR2 can be expressed on cancer cells, tumor infiltrating Treg and effector T cells. The present invention provides an antibody against a TNFR2 target for enhancing immunity against tumors.

### SUMMARY

In response to the shortcomings of the existing problems, the first purpose of the present invention is to provide an anti-TNFR2 antibody or antigen-binding fragment thereof.

The second purpose of the present invention is to provide a gene encoding the above-described anti-TNFR2 antibody or antigen-binding fragment thereof.

The third purpose of the present invention is to provide a vector comprising gene encoding the above-described anti-TNFR2 antibody or antigen-binding fragment thereof.

The fourth purpose of the present invention is to provide a host cell comprising a gene vector encoding the above-described anti-TNFR2 antibody or antigen-binding fragment thereof.

The fifth purpose of the present invention is to provide a method for expressing the above-described anti-TNFR2 antibody or antigen-binding fragment thereof.

The sixth purpose of the present invention is to provide a pharmaceutical conjugate comprising the above-described anti-TNFR2 antibody or antigen-binding fragment thereof.

The seventh purpose of the present invention is to provide a use of combination comprising the above-described anti-TNFR2 antibody or antigen-binding fragment thereof and a chemotherapy in the manufacture of a drug for the treatment of cancer or an autoimmune disease.

The eighth purpose of the present invention is to provide a use comprising the above-described anti-TNFR2 antibody or antigen-binding fragment thereof.

The ninth purpose of the present invention is to provide a pharmaceutical composition comprising the anti-TNFR2 antibody or antigen-binding fragment thereof.

The technical solution adopted by the present invention to solve its technical problems is:
1. An antibody or antigen-binding fragment thereof capable of specifically binding to human tumor necrosis factor receptor 2 (TNFR2), wherein it comprises heavy chains and light chains, and the heavy chain variable region comprises the heavy chain complementary determining regions CDR1, CDR2 and CDR3, and the light chain variable region comprises the light chain complementary determining regions CDR1, CDR2 and CDR3, wherein,
(01) CDR1, CDR2 and CDR3 of the heavy chain variable region consist of the amino acid sequences SEQ ID NO: 18, SEQ ID NO: 58 and SEQ ID NO: 20, respectively, and CDR1, CDR2 and CDR3 of the light chain variable region consist of SEQ ID NO: 22, SEQ ID NO: 23 and SEQ ID NO: 24, respectively;
(02) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 6, SEQ ID NO: 7 and SEQ ID NO: 8, respectively;
(03) CDR1, CDR2 and CDR3 of the heavy chain variable region consist of the amino acid sequences SEQ ID NO: 10, SEQ ID NO: 11 and SEQ ID NO: 12, respectively, and CDR1, CDR2 and CDR3 of the light chain variable region consist of SEQ ID NO: 14, SEQ ID NO: 15 and SEQ ID NO: 16, respectively;
(04) CDR1, CDR2 and CDR3 of the heavy chain variable region consist of the amino acid sequences SEQ ID NO: 18, SEQ ID NO: 19 and SEQ ID NO: 20, respectively, and CDR1, CDR2 and CDR3 of the light chain variable region consist of SEQ ID NO: 22, SEQ ID NO: 23 and SEQ ID NO: 24, respectively;
(05) CDR1, CDR2 and CDR3 of the heavy chain variable region consist of the amino acid sequences SEQ ID NO: 10, SEQ ID NO: 11 and SEQ ID NO: 12, respectively, and CDR1, CDR2 and CDR3 of the light chain variable region consist of SEQ ID NO: 27, SEQ ID NO: 15 and SEQ ID NO: 16, respectively;
(06) CDR1, CDR2 and CDR3 of the heavy chain variable region consist of the amino acid sequences SEQ ID NO: 18, SEQ ID NO: 55 and SEQ ID NO: 20, respectively, and CDR1, CDR2 and CDR3 of the light chain variable region consist of SEQ ID NO: 22, SEQ ID NO: 23 and SEQ ID NO: 24, respectively;
(07) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 28, SEQ ID NO: 7 and SEQ ID NO: 8, respectively;
(08) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 33, SEQ ID NO: 7 and SEQ ID NO: 8, respectively;
(09) CDR1, CDR2 and CDR3 of the heavy chain variable region consist of the amino acid sequences SEQ ID NO: 2, SEQ ID NO: 3 and SEQ ID NO: 36, respectively, and CDR1, CDR2 and CDR3 of the light chain variable region consist of SEQ ID NO: 6, SEQ ID NO: 7 and SEQ ID NO: 8, respectively;
(10) CDR1, CDR2 and CDR3 of the heavy chain variable region consist of the amino acid sequences SEQ ID NO: 18, SEQ ID NO: 55 and SEQ ID NO: 61, respectively, and CDR1, CDR2 and CDR3 of the light chain variable region consist of SEQ ID NO: 22, SEQ ID NO: 23 and SEQ ID NO: 24, respectively;
(11) CDR1, CDR2 and CDR3 of the heavy chain variable region consist of the amino acid sequences SEQ ID NO: 18, SEQ ID NO: 55 and SEQ ID NO: 64, respectively, and CDR1, CDR2 and CDR3 of the light chain variable region consist of SEQ ID NO: 22, SEQ ID NO: 23 and SEQ ID NO: 24, respectively;
(12) CDR1, CDR2 and CDR3 of the heavy chain variable region consist of the amino acid sequences SEQ ID NO: 18, SEQ ID NO: 58 and SEQ ID NO: 61, respectively, and CDR1, CDR2 and CDR3 of the light chain variable region consist of SEQ ID NO: 22, SEQ ID NO: 23 and SEQ ID NO: 24, respectively;
(13) CDR1, CDR2 and CDR3 of the heavy chain variable region consist of the amino acid sequences SEQ ID NO: 2, SEQ ID NO: 3 and SEQ ID NO: 36, respectively, and CDR1, CDR2 and CDR3 of the light chain variable region consist of SEQ ID NO: 28, SEQ ID NO: 7 and SEQ ID NO: 8, respectively;
(14) CDR1, CDR2 and CDR3 of the heavy chain variable region consist of the amino acid sequences SEQ ID NO: 2, SEQ ID NO: 3 and SEQ ID NO: 45, respectively, and CDR1, CDR2 and CDR3 of the light chain variable region consist of SEQ ID NO: 6, SEQ ID NO: 7 and SEQ ID NO: 8, respectively;
(15) CDR1, CDR2 and CDR3 of the heavy chain variable region consist of the amino acid sequences SEQ ID NO: 2, SEQ ID NO: 3 and SEQ ID NO: 45, respectively, and CDR1, CDR2 and CDR3 of the light chain variable region consist of SEQ ID NO: 28, SEQ ID NO: 7 and SEQ ID NO: 48, respectively; or
(16) CDR1, CDR2 and CDR3 of the heavy chain variable region consist of the amino acid sequences SEQ ID NO: 18, SEQ ID NO: 58 and SEQ ID NO: 64, respectively, and CDR1, CDR2 and CDR3 of the light chain variable region consist of SEQ ID NO: 22, SEQ ID NO: 23 and SEQ ID NO: 24, respectively.
2. The antibody or antigen-binding fragment thereof of scheme 1, wherein:
(01) the heavy chain variable region has the amino acid sequence shown in SEQ ID NO: 56, or a sequence having at least 80%, 85%, 90%, 91%, 92%, 93%, 94%, 95%, 96%, 97%, 98%, 99% or more identity to the amino acid sequence shown in SEQ ID NO: 56, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 56, and
the light chain variable region has the amino acid sequence shown in SEQ ID NO: 57, or a sequence having at least 80%, 85%, 90%, 91%, 92%, 93%, 94%, 95%, 96%, 97%, 98%, 99% or more identity to the amino acid sequence shown in SEQ ID NO: 57, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 56,
(02) the heavy chain variable region has the amino acid sequence shown in SEQ ID NO: 1, or a sequence having at least 80%, 85%, 90%, 91%, 92%, 93%, 94%, 95%, 96%, 97%, 98%, 99% or more identity to the amino acid sequence shown in SEQ ID NO: 1, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 1, and
the light chain variable region has the amino acid sequence shown in SEQ ID NO: 5, or a sequence having at least 80%, 85%, 90%, 91%, 92%, 93%, 94%, 95%, 96%, 97%, 98%, 99% or more identity to the amino acid sequence shown in SEQ ID NO: 5, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 5,
(03) the heavy chain variable region has the amino acid sequence shown in SEQ ID NO: 9, or a sequence having at least 80%, 85%, 90%, 91%, 92%, 93%, 94%, 95%, 96%, 97%, 98%, 99% or more identity to the amino acid sequence shown in SEQ ID NO: 9, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 9, and
the light chain variable region has the amino acid sequence shown in SEQ ID NO: 13, or a sequence having at least 80%, 85%, 90%, 91%, 92%, 93%, 94%, 95%, 96%, 97%, 98%, 99% or more identity to the amino acid sequence shown in SEQ ID NO: 13, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 13,
(04) the heavy chain variable region has the amino acid sequence shown in SEQ ID NO: 17, or a sequence having at least 80%, 85%, 90%, 91%, 92%, 93%, 94%, 95%, 96%, 97%, 98%, 99% or more identity to the amino acid sequence shown in SEQ ID NO: 17, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 17, and
the light chain variable region has the amino acid sequence shown in SEQ ID NO: 21, or a sequence having at least 80%, 85%, 90%, 91%, 92%, 93%, 94%, 95%, 96%, 97%, 98%, 99% or more identity to the amino acid sequence shown in SEQ ID NO: 21, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 21,
(05) the heavy chain variable region has the amino acid sequence shown in SEQ ID NO: 49, or a sequence having at least 80%, 85%, 90%, 91%, 92%, 93%, 94%, 95%, 96%, 97%, 98%, 99% or more identity to the amino acid sequence shown in SEQ ID NO: 49, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 49, and
the light chain variable region has the amino acid sequence shown in SEQ ID NO: 50, or a sequence having at least 80%, 85%, 90%, 91%, 92%, 93%, 94%, 95%, 96%, 97%, 98%, 99% or more identity to the amino acid sequence shown in SEQ ID NO: 50, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 50,
(06) the heavy chain variable region has the amino acid sequence shown in SEQ ID NO: 51, or a sequence having at least 80%, 85%, 90%, 91%, 92%, 93%, 94%, 95%, 96%, 97%, 98%, 99% or more identity to the amino acid sequence shown in SEQ ID NO: 51, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 51, and
the light chain variable region has the amino acid sequence shown in SEQ ID NO: 52, or a sequence having at least 80%, 85%, 90%, 91%, 92%, 93%, 94%, 95%, 96%, 97%, 98%, 99% or more identity to the amino acid sequence shown in SEQ ID NO: 52, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 52,
(07) the heavy chain variable region has the amino acid sequence shown in SEQ ID NO: 53, or a sequence having at least 80%, 85%, 90%, 91%, 92%, 93%, 94%, 95%, 96%, 97%, 98%, 99% or more identity to the amino acid sequence shown in SEQ ID NO: 53, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 53, and
the light chain variable region has the amino acid sequence shown in SEQ ID NO: 54, or a sequence having at least 80%, 85%, 90%, 91%, 92%, 93%, 94%, 95%, 96%, 97%, 98%, 99% or more identity to the amino acid sequence shown in SEQ ID NO: 54, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 54,
(08) the heavy chain variable region has the amino acid sequence shown in SEQ ID NO: 25, or a sequence having at least 80%, 85%, 90%, 91%, 92%, 93%, 94%, 95%, 96%, 97%, 98%, 99% or more identity to the amino acid sequence shown in SEQ ID NO: 25, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 25, and
the light chain variable region has the amino acid sequence shown in SEQ ID NO: 26, or a sequence having at least 80%, 85%, 90%, 91%, 92%, 93%, 94%, 95%, 96%, 97%, 98%, 99% or more identity to the amino acid sequence shown in SEQ ID NO: 26, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 26,
(09) the heavy chain variable region has the amino acid sequence shown in SEQ ID NO: 29, or a sequence having at least 80%, 85%, 90%, 91%, 92%, 93%, 94%, 95%, 96%, 97%, 98%, 99% or more identity to the amino acid sequence shown in SEQ ID NO: 29, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 29, and
the light chain variable region has the amino acid sequence shown in SEQ ID NO: 30, or a sequence having at least 80%, 85%, 90%, 91%, 92%, 93%, 94%, 95%, 96%, 97%, 98%, 99% or more identity to the amino acid sequence shown in SEQ ID NO: 30, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 30,
(10) the heavy chain variable region has the amino acid sequence shown in SEQ ID NO: 31, or a sequence having at least 80%, 85%, 90%, 91%, 92%, 93%, 94%, 95%, 96%, 97%, 98%, 99% or more identity to the amino acid sequence shown in SEQ ID NO: 31, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 31, and
the light chain variable region has the amino acid sequence shown in SEQ ID NO: 32, or a sequence having at least 80%, 85%, 90%, 91%, 92%, 93%, 94%, 95%, 96%, 97%, 98%, 99% or more identity to the amino acid sequence shown in SEQ ID NO: 32, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 32,
(11) the heavy chain variable region has the amino acid sequence shown in SEQ ID NO: 34, or a sequence having at least 80%, 85%, 90%, 91%, 92%, 93%, 94%, 95%, 96%, 97%, 98%, 99% or more identity to the amino acid sequence shown in SEQ ID NO: 34, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 34, and
the light chain variable region has the amino acid sequence shown in SEQ ID NO: 35, or a sequence having at least 80%, 85%, 90%, 91%, 92%, 93%, 94%, 95%, 96%, 97%, 98%, 99% or more identity to the amino acid sequence shown in SEQ ID NO: 35, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 35,
(12) the heavy chain variable region has the amino acid sequence shown in SEQ ID NO: 59, or a sequence having at least 80%, 85%, 90%, 91%, 92%, 93%, 94%, 95%, 96%, 97%, 98%, 99% or more identity to the amino acid sequence shown in SEQ ID NO: 59, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 59, and
the light chain variable region has the amino acid sequence shown in SEQ ID NO: 60, or a sequence having at least 80%, 85%, 90%, 91%, 92%, 93%, 94%, 95%, 96%, 97%, 98%, 99% or more identity to the amino acid sequence shown in SEQ ID NO: 60, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 60,
(13) the heavy chain variable region has the amino acid sequence shown in SEQ ID NO: 62, or a sequence having at least 80%, 85%, 90%, 91%, 92%, 93%, 94%, 95%, 96%, 97%, 98%, 99% or more identity to the amino acid sequence shown in SEQ ID NO: 62, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 62, and
the light chain variable region has the amino acid sequence shown in SEQ ID NO: 63, or a sequence having at least 80%, 85%, 90%, 91%, 92%, 93%, 94%, 95%, 96%, 97%, 98%, 99% or more identity to the amino acid sequence shown in SEQ ID NO: 63, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 63,
(14) the heavy chain variable region has the amino acid sequence shown in SEQ ID NO: 65, or a sequence having at least 80%, 85%, 90%, 91%, 92%, 93%, 94%, 95%, 96%, 97%, 98%, 99% or more identity to the amino acid sequence shown in SEQ ID NO: 65, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 65, and
the light chain variable region has the amino acid sequence shown in SEQ ID NO: 66, or a sequence having at least 80%, 85%, 90%, 91%, 92%, 93%, 94%, 95%, 96%, 97%, 98%, 99% or more identity to the amino acid sequence shown in SEQ ID NO: 66, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 66,
(15) the heavy chain variable region has the amino acid sequence shown in SEQ ID NO: 37, or a sequence having at least 80%, 85%, 90%, 91%, 92%, 93%, 94%, 95%, 96%, 97%, 98%, 99% or more identity to the amino acid sequence shown in SEQ ID NO: 37, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 37, and
the light chain variable region has the amino acid sequence shown in SEQ ID NO: 38, or a sequence having at least 80%, 85%, 90%, 91%, 92%, 93%, 94%, 95%, 96%, 97%, 98%, 99% or more identity to the amino acid sequence shown in SEQ ID NO: 38, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 38,
(16) the heavy chain variable region has the amino acid sequence shown in SEQ ID NO: 39, or a sequence having at least 80%, 85%, 90%, 91%, 92%, 93%, 94%, 95%, 96%, 97%, 98%, 99% or more identity to the amino acid sequence shown in SEQ ID NO: 39, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 39, and
the light chain variable region has the amino acid sequence shown in SEQ ID NO: 40, or a sequence having at least 80%, 85%, 90%, 91%, 92%, 93%, 94%, 95%, 96%, 97%, 98%, 99% or more identity to the amino acid sequence shown in SEQ ID NO: 40, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 40,
(17) the heavy chain variable region has the amino acid sequence shown in SEQ ID NO: 41, or a sequence having at least 80%, 85%, 90%, 91%, 92%, 93%, 94%, 95%, 96%, 97%, 98%, 99% or more identity to the amino acid sequence shown in SEQ ID NO: 41, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 41, and
the light chain variable region has the amino acid sequence shown in SEQ ID NO: 42, or a sequence having at least 80%, 85%, 90%, 91%, 92%, 93%, 94%, 95%, 96%, 97%, 98%, 99% or more identity to the amino acid sequence shown in SEQ ID NO: 42, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 42,
(18) the heavy chain variable region has the amino acid sequence shown in SEQ ID NO: 43, or a sequence having at least 80%, 85%, 90%, 91%, 92%, 93%, 94%, 95%, 96%, 97%, 98%, 99% or more identity to the amino acid sequence shown in SEQ ID NO: 43, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 43, and
the light chain variable region has the amino acid sequence shown in SEQ ID NO: 44, or a sequence having at least 80%, 85%, 90%, 91%, 92%, 93%, 94%, 95%, 96%, 97%, 98%, 99% or more identity to the amino acid sequence shown in SEQ ID NO: 44, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 44,
(19) the heavy chain variable region has the amino acid sequence shown in SEQ ID NO: 46, or a sequence having at least 80%, 85%, 90%, 91%, 92%, 93%, 94%, 95%, 96%, 97%, 98%, 99% or more identity to the amino acid sequence shown in SEQ ID NO: 46, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 46, and
the light chain variable region has the amino acid sequence shown in SEQ ID NO: 47, or a sequence having at least 80%, 85%, 90%, 91%, 92%, 93%, 94%, 95%, 96%, 97%, 98%, 99% or more identity to the amino acid sequence shown in SEQ ID NO: 47, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 47, or
(20) the heavy chain variable region has the amino acid sequence shown in SEQ ID NO: 67, or a sequence having at least 80%, 85%, 90%, 91%, 92%, 93%, 94%, 95%, 96%, 97%, 98%, 99% or more identity to the amino acid sequence shown in SEQ ID NO: 67, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 67, and
the light chain variable region has the amino acid sequence shown in SEQ ID NO: 68, or a sequence having at least 80%, 85%, 90%, 91%, 92%, 93%, 94%, 95%, 96%, 97%, 98%, 99% or more identity to the amino acid sequence shown in SEQ ID NO: 68, or an amino acid sequence having one or more (preferably 1, 2, 3, 4, 5, 6, 7, 8, 9, 10) conserved amino acid mutations (preferably substitutions, insertions, or deletions) in comparison to the amino acid sequence of SEQ ID NO: 68.
3. The antibody or antigen-binding fragment thereof of any one of schemes 1-2, which binds to TNFR2 of Treg and can kill Treg, with a kill rate of at least 10%, 20%, 30%, 40%, 80%, 85%, 90%, 91%, 92%, 93%, 94%, 95%, 96%, 97%, 98%, 99%, 96%, 97%, 98%, 99% or 100%.
4. The antibody or antigen-binding fragment thereof of any one of schemes 1-2, which binds to TNFR2 of Teff and promotes the proliferation and differentiation of Teff cells.
5. The antibody or antigen-binding fragment thereof of any one of schemes 1-2, which binds to TNFR2 of Teff and promotes the bioactivity of Teff.
6. The antibody or antigen-binding fragment thereof of any one of schemes 1-2, which has a dissociation constant KD of less than 10 nM with TNFR2.
7. The antibody or antigen-binding fragment thereof of any one of schemes 1-2, which has a dissociation constant KD of less than 1 nM with TNFR2.
8. A polynucleotide encoding the antibody or antigen-binding fragment thereof of any one of schemes 1-2.
9. A recombinant vector, transgenic cell line, phage, recombinant bacterium or viral vector comprising the polynucleotide of scheme 8.
10. Use of the recombinant vector, transgenic cell line, phage, recombinant bacterium or viral vector of scheme 9 in the preparation of an anti-TNFR2 antibody.
11. An isolated host cell comprising the recombinant vector, transgenic cell line, phage, recombinant bacterium or viral vector of scheme 10.
12. The host cell of scheme 11, wherein the host cell is a prokaryotic cell.
13. The host cell of scheme 11, wherein the host cell is a eukaryotic cell.
14. The host cell of scheme 13, wherein the eukaryotic cell is a mammalian cell.
15. The host cell of scheme 14, wherein the mammalian cell is a CHO cell.
16. A method of antibody expression, wherein the method uses the recombinant vector, transgenic cell line, phage, recombinant bacterium or viral vector of scheme 9 to express an antibody protein in the host cell of any one of schemes 11-15.
17. The antibody or antigen-binding fragment thereof of any one of schemes 1-2, wherein the antibody or antigen-binding fragment thereof comprises murine, human-mouse chimeric, humanized or fully human.
18. The antibody or antigen-binding fragment thereof of any one of schemes 1-2, wherein the antibody or antigen-binding fragment thereof is selected from the group consisting of: monoclonal antibody or antigen-binding fragment thereof, polyclonal antibody or antigen-binding fragment thereof, humanized antibody or antigen-binding fragment thereof, primateized antibody or antigen-binding fragment thereof, bispecific antibody or antigen-binding fragment thereof, multispecific antibody or antigen-binding fragment thereof, dual variable immunoglobulin structural domain, monovalent antibody or antigen-binding fragment thereof, chimeric antibody or antigen-binding fragment thereof, single-chain Fv molecule (scFv), bispecific antibody, trispecific antibody, nanobody, antibody-like protein scaffold, structural domain antibody, Fv fragment, Fab fragment, F(ab′)2 molecule, and tandem scFv.
19. The antibody or antigen-binding fragment thereof of any one of schemes 1-2, wherein the antibody is a monoclonal antibody.
20. Use of the antibody or antigen-binding fragment thereof of any one of schemes 1-2 in the preparation of a drug comprising the antibody or antigen-binding fragment thereof and a chemotherapy for the treatment of a tumor in a human patient, wherein the antibody and the chemotherapy are formulated to provide a therapeutic effect that is greater than the sum of the respective effects of the reagents.
21. An antibody-drug conjugate, wherein the antibody or antigen-binding fragment thereof of any one of schemes 1-2 is optionally coupled to a therapeutic agent via a linker.
22. The antibody-drug conjugate of scheme 21, wherein the therapeutic agent is a cytotoxic agent.
23. A pharmaceutical composition comprising the antibody or antigen-binding fragment thereof of any one of schemes 1-2 and a pharmaceutically acceptable carrier, diluent or excipient.
24. The pharmaceutical composition of scheme 23, wherein the composition further comprises an additional therapeutic agent.
25. The pharmaceutical composition of scheme 24, wherein the additional therapeutic agent is an immunotherapeutic agent.
26. Use of the antibody or antigen-binding fragment thereof of any one of schemes 1-2 in the following (a), (b), (c), (d) or (e):
(a) preparation of a drug that induces apoptosis in Treg cells;
(b) preparation of a drug that targets tumor cell killing;
(c) preparation of a drug that blocks the TNF-TNFR2 tumor signaling pathway;
(d) preparation of a drug that promotes the proliferation, differentiation and/or biological activity of Teff cells; or
(e) preparation of a drug for the treatment of autoimmune diseases.
27. The use of scheme 26, wherein the tumor is selected from: ovarian cancer, melanoma, prostate cancer, intestinal cancer, gastric cancer, oesophageal cancer, breast cancer, lung cancer, renal cancer, pancreatic cancer, uterine cancer, liver cancer, bladder cancer, cervical cancer, oral cavity cancer, brain cancer, testicular cancer, skin cancer, colorectal cancer, malignant glioma, and thyroid cancer.

The present invention provides an antibody or antigen-binding fragment thereof that binds specifically to TNFR2 with high affinity, and the antibody can mediate killing activity against Treg cells, mediate proliferative and activating effects on CD8+ T cells, and have excellent anti-tumor efficacy.

The following definitions are provided to assist in understanding the invention set forth herein.

Provided herein are isolated antibodies, including murine, chimeric, and human antibodies, which specifically bind to a particular epitope on TNFR2 (e.g., human TNFR2). Also provided herein are methods of preparing antibodies, pharmaceutical conjugates comprising the antibodies or antigen-binding fragments thereof of the present invention, pharmaceutical compositions, and combination therapies with other therapeutic agents, and the preparation of a drug for the treatment of a wide range of diseases using the antibodies or antigen-binding fragments thereof of the present invention.

The BMK herein comprises BMK2 and BMK6. The sequences are obtained from: SEQ ID NO: 3 and SEQ ID NO: 4 in the Patent WO2017083525A1; and SEQ ID NO: 150 and SEQ ID NO: 151 in the Patent WO2020061210A1, respectively.

The terms “Tumor Necrosis Factor Receptor 2”, “TNFR2”, “CD120b”, “p75”, “p75TNFR”, “p80TNF-α receptor”, “TBPII”, “TNFBR”, “TNFR1B”, “TNF-R75” and “TNFR80” are used interchangeably herein, and include all family members, mutants, alleles, fragments and species. TNFR2 mediates TNFα activity in conjunction with TNFR1. TNFR1 is a 55 kD membrane-bound protein, whereas TNFR2 is a 75 kD membrane-bound protein. TNFR2 regulates TNFα binding to TNFR1, and therefore regulates the level of TNFα necessary to stimulate NF-kB action. TNFR2 can also be cleaved (or selectively spliced) by metalloproteinases to generate soluble receptors that maintain affinity for TNFα.

The present invention also provides a composition. It contains the antibody or an active fragment thereof of the invention, and a pharmaceutically acceptable carrier. Typically, these substances may be formulated in a non-toxic, inert and pharmaceutically acceptable aqueous carrier medium, the pH of which may vary with the nature of the substance being formulated and the condition to be treated. The formulated pharmaceutical compositions can be administered by conventional routes, which include, but are not limited to: intratumoral, intraperitoneal, intravenous, or topical administration.

The pharmaceutical compositions of the present invention can be used to bind to TNFR2 protein molecules directly and thus can be used to treat tumors. In addition, other therapeutic agents may be used concurrently.

The pharmaceutical compositions of the present invention contain a safe and effective amount (e.g., 0.001-99.999 wt %, preferably 0.01-90 wt %, more preferably 0.1-80 wt %) of the above-described antibodies or binding fragments thereof (or conjugates thereof) of the present invention as well as a pharmaceutically acceptable carrier or excipient. Such carriers include, but are not limited to: saline, buffer, glucose, water, glycerol, ethanol, and combinations thereof. The pharmaceutical formulation should be compatible with the mode of administration. The pharmaceutical compositions of the present invention may be made in the form of an injection, e.g., prepared by conventional methods using saline or an aqueous solution containing dextrose and other excipients. The pharmaceutical compositions such as injections and solutions are preferably manufactured under sterile conditions. The active ingredient is administered in a therapeutically effective amount. In addition, the peptides of the present invention may be used with other therapeutic agents.

The antibody of the present invention may be used alone or in combination or conjugation with a detectable marker (for diagnostic purposes), a therapeutic agent, a PK (protein kinase) modifying portion, or any combination of these. Detectable markers for diagnostic purposes include but are not limited to: fluorescent or luminescent markers, radiolabeled markers, MRI (magnetic resonance imaging) or CT (computerized tomography) contrast agents, or enzymes that are capable of producing a detectable product. Therapeutic agents that can be bound or coupled to antibodies of the present invention include, but are not limited to: 1. radionuclides; 2. biotoxins; 3. cytokines such as IL-2, etc.; 4. gold nanoparticles/nanorods; 5. viral particles; 6. liposomes; 7. magnetic nanoparticles; 8. pre-drug-activating enzymes (e.g., DT-diaphorase (DTD) or biphenyl hydrolysate-like proteins (BPHL)); 9. chemotherapeutic agents (e.g., cisplatin) or any form of nanoparticles, etc.

In this description, “antibody” means a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells. Alternatively, they may be partially or completely synthesized by using techniques such as genetic recombination. Preferred antibody include, for example, antibody to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Among these isotypes, the antibody of the present invention may include IgG1, IgG2, IgG3, and IgG4.

The term “antibody” or “immunoglobulin” used interchangeably herein include an entire antibody and any antigen-binding fragment thereof (antigen-binding portion) or a single chain thereof. “Antibody” includes at least one heavy (H) chain and one light (L) chain. For example, in naturally existing IgG, these heavy and light chains are linked to each other by disulfide bonds and there are two pairs of paired heavy and light chains. These two pairs of paired heavy and light chains are also interconnected by disulfide bonds, and each heavy chain consists of a heavy chain variable region (abbreviated herein as VH) and a heavy chain constant region, and the heavy chain constant region consists of three structural domains, CH1, CH2 and CH3. The light chain consists of a light chain variable region (abbreviated herein as VL) and a light chain constant region, and the light chain constant region consists of one structural domain CL. The VH and VL regions can be further subdivided into highly variable regions called complementary determining regions (CDRs), scattered with more conserved regions called framing regions (FRs) or joining regions (J) (JH or JL in heavy and light chains, respectively), each VH and VL consists of three CDRs, three FRs and one J structural domain, which are arranged from the amino terminus to the carboxyl terminus in the following order: FR1, CDR1, FR2, CDR2, FR3, CDR3, J. The variable regions of the heavy and light chains bind to antigen. The constant region of the antibody may mediate the binding of immunoglobulins to host tissues or factors, including various cells of the immune system (e.g., effector cells) or humoral factors, such as the first part of the classical complement system (C1q). Fragments of full-length antibodies have been shown to perform the antigen-binding function of the antibody. Examples of binding fragments represented as antigen-binding portions or antibody fragments include (i) a Fab fragment, a monovalent fragment comprising: (ii) an F(ab′)2 fragment, a bivalent fragment comprising two Fab fragments linked by a disulfide bond in the hinge region; (iii) an Fd fragment comprising the VH and CH1 domains; (iv) an Fv fragment comprising the VL and VH structural domains of the single arm of the antibody, (v) a dAb comprising the VH and VL structural domains; (vi) a dAb fragment comprising the VH structural domains; (vii) a dAb comprising the VH or VL structural domains; (viii) an isolated complementary determining region (CDR) or (ix) a combination of two or more isolated CDRs, optionally linked by a synthetic linker. In addition, although the two structural domains of the Fv fragment, VL and VH, are encoded for separate genes, they can be linked using recombinant methods via a synthetic junction that makes them into a protein chain in which the VL and VH regions pair to form a monovalent molecule (this immunoglobulin fragment is the single chain homologue of this single chain antibody also intended to be included within the term “antibody”). Antibody fragments are obtained using conventional techniques known to those skilled in the art and screened for the utility of the fragment in the antibody. The antigen-binding portion may be produced by recombinant DNA technology or by enzymatic or chemical cleavage of intact immunoglobulins. The numbering of amino acid positions (e.g., amino acid residues in the Fc region) and target regions, e.g., CDRs, in the antibodies described herein are performed using the Kabat system.

As used herein, “isotype” refers to an antibody class encoded by a heavy chain constant region gene (e.g., IgG1, IgG2, IgG3, IgG4, IgM, IgA1, IgA2, IgD and IgE antibody).

The term “recombinant antibody” means an antibody prepared, expressed, produced or isolated by recombinant means. For example, (a) antibody genes isolated from or hybridomas prepared from animals (e.g., mice) transgenic or transchromosomal for immunoglobulin genes (e.g., human immunoglobulin). (b) an antibody isolated from a host cell that has been transformed to express the antibody, e.g., from a transfected tumor. (c) an antibody isolated from a combined combinatorial antibody library (e.g., comprising a human antibody sequence) using phage display. (d) preparing, expressing, generating, or isolating antibodies by any other means of splicing an immunoglobulin gene sequence (e.g., a human immunoglobulin gene) into other DNA sequences. Such recombinant antibodies may have variable and constant regions derived from human germline immunoglobulin sequences. However, in some embodiments, such recombinant human antibodies may be subjected to in vitro mutagenesis; the VH and VL regions of the recombinant antibody are sequences derived from and related to human germline VH and VL sequences that may not be naturally present in the in vivo human antibody germline library.

The term “chimeric immunoglobulin” or “chimeric antibody” refers to an immunoglobulin or antibody whose variable region is derived from a first species and whose constant region is derived from a second species. The chimeric immunoglobulin or antibody may be constructed, for example by genetic engineering, from immunoglobulin gene segments belonging to different species.

The term “humanized antibody” means an antibody comprising at least one humanized antibody chain (i.e. at least one humanized light or heavy chain). The term “humanized antibody chain” (i.e. “humanized immunoglobulin chain”) refers to an antibody chain (i.e. light or heavy chain, respectively) having a variable region comprising a substantially variable framework region and complementarity determination of the human antibody. Complementary determining regions (CDRs) substantially derived from a non-human antibody (e.g., at least one CDR, two CDRs, or three CDRs). In some embodiments, the humanized antibody chain further comprises constant regions (e.g., in the case of a light chain, one constant region or a portion thereof, in the case of a heavy chain, preferably three constant regions).

As used herein, the term “human antibody” refers to an antibody comprising a variable region, wherein both the constitutive region and the CDR region are derived from human immunoglobulin sequences. In addition, if the antibody comprises a constant region, the constant region is also derived from a human germline immunoglobulin sequence. The human antibody may include amino acid residues not encoded by the human germline immunoglobulin sequence (e.g., mutations introduced in vitro by random site-specific mutagenesis or in vivo somatic mutation).

The human antibody may have at least one or more amino acids substituted by amino acid residues, the amino acid residues being, for example, activity-enhancing amino acid residues not encoded by a human immunoglobulin sequence. Typically, the human antibody may have up to twenty positions that are substituted with amino acid residues that are not part of the human germline immunoglobulin sequence. In particular embodiments, these substitutions are within the CDR region. In some other embodiments, these substitutions are within the framework region.

As used herein, the term “multispecific” or “multifunctional antibody” is an artificial hybrid antibody having multiple different binding sites. Bispecific antibodies can be generated by a variety of methods, including fusion hybridomas or ligation of Fab′ fragments.

As used herein, the term “isolated” refers to antibody that is substantially free of other antibodies having different antigenic specificities. In addition, isolated antibody is typically substantially free of other cellular and/or chemical substances.

As used herein, the term “Fc region”, “Fc structural domain” or “Fc” refers to the C-terminal region of the heavy chain of an antibody. Thus, the Fc region comprises the constant region of the antibody, but does not include the first constant region immunoglobulin structural domain (e.g., CH1 or CL).

As used herein, the term “antigen” is an antibody-bound entity (e.g., a protein entity or peptide), such as TNFR2.

As used herein, the terms “specific binding” and “selective binding” mean that the antibody exhibits appreciable affinity for a particular antigen or epitope and does not normally exhibit appreciable cross-reactivity with other antigens or epitopes. “Appreciable” or preferred binding includes binding at a KD of 10⁻⁷, 10⁻⁸, 10⁻⁹ or 10⁻¹⁰M or more preferably. The KD (affinity constant) for antibody-antigen interactions represents the concentration of antibody at which 50% of the antibody and antigen molecules are bound together. Thus, at a suitable fixed antigen concentration, 50% of a higher affinity (i.e. stronger) antibody binds to the antigen molecule at a lower antibody concentration compared with the antibody concentration required to achieve the same percentage of binding with a lower affinity antibody. Thus, a lower KD value indicates a higher (stronger) affinity. As used herein, a “better” affinity is stronger than its affinity and has a lower value of KD of 10⁻⁷ M, and thus a better affinity compared with a KD of 10⁻⁶ M. It is generally preferred to have a KD value of less than 10⁻⁷ M, and therefore preferably greater than 10⁻⁸ M. Intermediate values as described herein may also be considered, and the preferred binding affinity may be expressed as a range of affinities, e.g., from 10⁻⁷ to 10⁻¹² M for the anti-TNFR2 antibodies disclosed herein, and more preferably from 10⁻⁸ to 10⁻¹² M. Antibodies that “do not exhibit significant cross-reactivity” or “do not bind with physiologically relevant affinity” are antibodies that do not significantly bind to the antibody. Off-target antigens (e.g., non-TNFR2 proteins) or epitopes. Specific or selective binding can be determined according to any art of the art. Recognized methods for determining such binding include, for example, according to Scatchard analysis, biomolecule interaction analysis, biofilm layer interferometry and/or competitive (competitive) binding assays.

As used herein, the term “epitope” means an antigenic determinant cluster in an antigen and refers to an antigenic site bound by a structural domain of an antigen-binding molecule comprising an antibody variable region as disclosed in this specification. Thus, an epitope can be defined, for example, on the basis of its structure. Alternatively, the epitope may be defined based on the antigen-binding activity in the antigen-binding molecule that recognizes the epitope. When the antigen is a peptide or polypeptide, the epitope may be designated by the amino acid residues forming the epitope. Alternatively, when the epitope is a sugar chain, the epitope may be defined by its specific sugar chain structure.

A linear epitope is an epitope containing a primary amino acid sequence for which it is recognized. Such linear epitopes typically contain at least three, most commonly at least five, such as about 8 to 10 or 6 to 20 amino acids in their particular sequence.

In contrast to linear epitopes, “conformational epitopes” are epitopes in which the primary amino acid sequence containing the epitope is not the sole determinant of the recognized epitope (e.g. the primary amino acid sequence of the conformational epitope is not necessarily recognized by the epitope-qualifying antibody). A conformational epitope may contain a greater number of amino acids than a linear epitope. Conformational epitopes recognize the three-dimensional structure of the peptide or protein recognized by the antibody. For example, when a protein molecule folds and forms a three-dimensional structure, the amino acids and/or peptide backbone forming the conformational epitope become aligned and the epitope can be recognized by the antibody. Methods for determining epitope conformation include, for example, but are not limited to, X-ray crystallography, two-dimensional nuclear magnetic resonance, site-specific spin labelling and electron paramagnetic resonance.

As used herein, the term “vector” refers to a nucleic acid molecule capable of transporting another nucleic acid to which it has been attached. One type of vector is a “plasmid”, which refers to a circular double-stranded DNA loop. Other DNA fragments can be attached to it. Another type of vector is the viral vector, in which additional DNA fragments can be attached to the viral genome. Some vectors are capable of replicating autonomously in the host cell into which they are introduced (e.g., bacterial vectors with replicating bacterial origins and free-living mammalian vectors). Other vectors (e.g., non-exogenous mammalian vectors) can be integrated into the genome of the host cell and then introduced into the host cell, thereby replicating with the host genome. In addition, some vectors are capable of directing the expression of the genes they express These vectors are referred to herein as “recombinant expression vectors” (or simply “expression vectors”). Typically, expression vectors useful in recombinant DNA technology are usually in the form of plasmids. The terms “plasmid” and “vector” are used interchangeably. However, other forms of expression vectors with equivalent functions are also contemplated, such as viral vectors (e.g., replication-defective retroviruses, adenoviruses and adeno-associated viruses).

As used herein, the term “inhibition” refers to any statistically significant reduction in biological activity, including partial and complete blockage of that activity. For example, “inhibition” may denote a statistically significant decrease of about 10%, 20%, 30%, 40%, 50%, 60%, 70%, 80%, 90%, 95%, 96%, 97%, 98%, 99% or 100% of the biological activity.

As used herein, the term “activation” refers to any statistically significant activation of the biological activity of a cell, e.g., “activation” may denote a statistically significant increase of about 1%, 10%, 20%, 30%, 40%, 50%, 60%, 70%, 80%, 90%, 100%, 200%, 500%, 1,000%, 10,000%, and more of biological activity.

As used herein, the phrase “inhibits binding of TNFR2 ligand to TNFR2” means that the antibody statistically significantly reduces the ability of the TNFR2 ligand (e.g., TNFα) to bind to TNFR2 relative to the absence of the TNFR2 antibody. In other words, in the presence of an antibody, the amount of TNFR2 ligand that binds to TNFR2 is statistically significantly reduced relative to the control (no antibody). In the presence of an anti-TNFR2 antibody as disclosed herein, the amount of TNFR2 ligand binding to TNFR2 can be reduced by at least about 10%, or at least about 20%, or at least about 30%, or at least about 40%, or at least about 50%, or at least about 60%, or at least about 70%, or at least about 80%, or at least about 90%, or about 100%. The reduction in TNFR2 ligand binding can be measured using techniques accepted in the art that measure the level of binding of labeled TNFR2 ligand (e.g., radiolabeled TNFα) to cells expressing TNFR2 in the presence or absence of (control) antibody.

As used herein, the term “inhibition of tumor growth” includes any measurable reduction in tumor growth, e.g., an inhibition of tumor growth of at least about 10%, e.g., at least about 20%, at least about 30%, at least about 40%, at least about 50%, at least about 60%, at least about 70%, at least about 80%, at least about 90%, at least about 99%, or about 100%.

As used herein, the term “treatment” refers to the therapeutic or prophylactic measures described herein. Methods of “treatment” are intended for administration to a subject or subjects susceptible to tumors or cancers. An anti-TNFR2 antibody (e.g., an anti-human TNFR2 antibody) is described herein for the purpose of preventing, curing, delaying, or alleviating one or more symptoms of a disease or condition or recurrent disease or condition, or for the purpose of prolonging the survival of a subject for a prolonged period of time in the absence of such treatment.

As used herein, the term “variable fragment (Fv)” refers to the smallest unit of an antibody-derived antigen-binding structural domain, which consists of a pair of antibody light chain variable regions (VL) and antibody heavy chain variable regions (VH). In 1988, Skerra and Pluckthun found that homologous and active antibodies could be prepared from the periplasmic fraction of E. coli by inserting an antibody gene downstream of a bacterial signal sequence and inducing expression of the gene in E. coli. In Fv prepared from the periplasmic fraction, VH binds to VL in an antigen-binding manner.

Herein, the Fv preferably comprises, for example, a pair of Fvs acting as antigen-binding molecules, etc., comprising: scFv, a single-chain antibody, and sc(Fv)2.

As used herein, the terms “scFv”, “single chain antibody” and “sc(Fv)2” all refer to an antibody fragment of a single polypeptide chain containing variable regions derived from the heavy and light chains, but no constant regions. Typically, single-chain antibodies also contain a polypeptide junction between the VH and VL structural domains, which enables the formation of the desired structure thought to permit antigen binding.

As used herein, the term “CHOK1-hTNFR2” refers to a cell constructed by certain techniques. Specific construction methods include, but are not limited to, the following methods. Detailed steps: (1) gene synthesis and molecular construction: the sequence of human TNFR2 protein was codon optimized and synthesized by GENEWIZ. The synthesized gene was further subcloned into the modified pSBbi-GB vector. (2) Transient transfection: 1.0×10⁶ CHOK1 cells with higher than 95% survivability were prepared in T25 culture flasks one day prior to transfection, and transfection was initiated at 70%-90% confluence. The target gene was mixed with the transposase plasmid pCMV(CAT)T7-SB100 in a 9:1 ratio, followed by the addition of the Lipofectamine® 2000 DNA transfection reagent mix, and then the cell culture medium was added. The cells were kept in a 37° C. thermostat while the CO₂ concentration was maintained at 8%. After 48 hours of incubation, 2×10⁵ cells were firstly detached by trypsin and then centrifuged at 1,500 rpm, 4° C. for 4 minutes for FACS detection of TNFR2 expression levels. (3) Stable cell pool and cell line production: After transient transfection with FACS to confirm TNFR2 expression, the cell pool was further cultured in a medium containing blasticidin for stable cell pool selection. The concentration of blasticidin is 10 μg/mL. Change the culture medium every 2-3 days. After recovering from 2 to 3 weeks of antibiotic selection, a stable cell pool will be generated. Further generation of stable single cell lines through BD FACS Melody sorting. Firstly, count the cells and measure their vitality. After incubation with primary and secondary antibodies, individual cells were sorted into 96 well plates and cultured in an incubator until clones could be observed. After FACS detection, the highly expressed monoclonal clones were amplified and stored in the library. (4) FACS assay: transiently transfected cells were transferred to 96-well U-bottom plates (Corning-3799) at a density of 2×10⁵ cells/well. Anti-TNFR2 antibody was diluted at 2 μg/mL in 2% FBS/1×PBS at 100 μL per well and then incubated for 1 hour at 4° C. Cells were washed twice and resuspended in 100 μL of 2% BSA/1×PBS. Secondary antibody (goat anti-human IgGFc-Alexa647) was diluted 1:500 in 2% FBS/1×PBS, 100 μL per well, followed by incubation at 4° C. for 30 minutes. Cells were washed twice and resuspended in 100 μL of 2% FBS/1×PBS. Fluorescence was measured by flow cytometry (BD Canto II) and analyzed by FlowJo. (5) Stability assay: cell libraries or cell lines passaged for more than 3 weeks after P1 are used for stability assay. FACS is used for stability confirmation.

As used herein, the terms “Treg”, “Treg cell” or “regulatory T cell” are sometimes referred to as suppressor T cells, wherein, the expression of the biomarkers CD4, FOXP3 and CD25, and represent subsets of T cells that regulate the immune system, maintain tolerance to self-antigens and prevent autoimmune diseases. Tregs are immunosuppressive and generally inhibit or downregulate the induction and proliferation of effector T (Teff) cells. Tregs can develop in the thymus (so-called CD4+Foxp3+ “natural” Treg) or differentiate from naive CD4+ T cells in the periphery, e.g. after exposure to TGFβ or retinoic acid.

As used herein, the term “Teff”, “Teff cell” or “effector T cell” refers to a cell that is formed by the proliferation and differentiation of T cells after they have been stimulated by an antigen. Effector T cells have the function of releasing lymphokines, and in this process, a small proportion of T cells become memory T cells. Effector T cells contact with target cells and stimulate granule exocytosis, the released perforin forms pores on the surface of the target cell through polymerization, thus mediating the killing effect, and its target cell death process is similar to apoptosis. At the same time, effector T cells can also release immunoreactive substances-lymphokines, such as interleukins, interferons and so on.

### BRIEF DESCRIPTION OF THE DRAWINGS

FIG. 1 shows a schematic diagram of FACS binding of rat antibodies to hTNFR2;

FIG. 2 shows a schematic diagram of the ligand-blocking ELISA assay for rat antibodies binding to hTNFR2;

FIG. 3 shows a schematic diagram of the anti-TNFR2 antibody-mediated killing activity against Treg cells;

FIG. 4 shows a schematic diagram of the anti-TNFR2 antibody-mediated proliferative effect on CD8+ T cells;

FIG. 5 shows a schematic diagram of the anti-TNFR2 antibody-mediated activation of CD8+ T cells;

FIG. 6 shows a schematic diagram of in vivo antitumor efficacy evaluation of chimeric antibodies;

FIG. 7 shows the diagram of in vivo drug efficacy experiments-tumor volume changes in the mouse MC38 transplanted tumor mouse model;

FIG. 8 shows the diagram of in vivo drug efficacy experiments-body weight changes in the mouse MC38 transplanted tumor mouse model;

FIG. 9 shows the diagram of in vivo drug efficacy experiments-tumor volume changes in the mouse B16-F10 transplanted tumor mouse model.

### DETAILED DESCRIPTION

The following embodiments facilitate a better understanding of the invention, but do not limit the invention. The experimental methods in the following embodiments are conventional methods, if not otherwise specified. Unless otherwise stated, the test materials used in the following embodiments are purchased from a conventional biochemical reagent shop.

### Example 1 Animal Immunization

Recombinant human TNFR2-His Tag protein (Sino, Cat: 10417-H08H) was used as the immunogen to immunize SD rats, and 3-4 rats could be immunized at the same time in order to avoid poor response or apoptosis during immunization. The negative serum was taken one day in advance, and 60 kg of recombinant human TNFR2-His Tag protein fully emulsified with Fuchs' complete adjuvant was injected into multiple points by subcutaneous, intraperitoneal and plantar immunization for the first immunization. On the 23rd day, 40 kg of recombinant human TNFR2-His Tag protein fully emulsified with Fuchs' complete adjuvant was injected into multiple points by subcutaneous and plantar immunization for the second immunization, and 40 g of immunogen was injected in the same way as the second immunization every two weeks for the third, fourth, fifth, sixth and seventh immunization. After 30 days, antiserum titres were assessed by testing serum collected from tail bloodletting in recombinant human TNFR2-His Tag protein-coated ELISA plates at different dilutations from 1:100 to 1:1,000,000. When the titre results meet the requirements and anti-human TNFR2 antibodies are detected at dilutations >1:1,000,000, rat spleens and lymph nodes can be harvested for cell fusion.

### Example 2 Cell Fusion

Myeloma cells SP2/0 (ATCC) were passaged one day before fusion so that the cells were in the logarithmic growth phase at the time of the experiment. Before fusion, the cells were collected in a centrifuge tube, and centrifuged at 1000 rpm for 5 minutes. The supernatant was discarded and 10 mL of DMEM complete medium was added to mix the cells well for use. Spleen and lymph node cells used in the experiment were taken from SD rats immunized with recombinant human TNFR2-His Tag protein for seven times. The rats were killed before fusion, and spleen and lymph nodes were taken. The cells were pressed and ground through the net by adding 10 mL of DMEM complete medium. The obtained cell suspension was centrifuged at 2,000 rpm for 8 minutes, and then the supernatant was discarded and added to the DMEM complete medium to mix the cells well for use. The feeder cells used in the fusion selection culture were taken from macrophages in the peritoneal cavity of unimmunized animals. Before fusion, the collected macrophage suspension was centrifuged at 100 rpm for 8 minutes. The supernatant was discarded and added to 25 mL of HAT selection medium to mix well, then dispensed into two 24-well culture plates to assist the growth of new hybrid B-lymphocyte hybridomas.

B lymphocytes and myeloma cells were mixed at 1:2, and the suspension was centrifuged at 1000 rpm for 8 minutes. The precipitate was taken, and washed twice with the electrofusion solution. The precipitate was taken, and the electrofusion solution was added to 1.2 mL. 0.4 mL of cell suspension was injected into each of the three multi-electrode small cuvettes. The following electric field conditions were applied to each electrode cuvette in turn: the AC electric field sinusoidal signal frequency of dielectric electrophoresis was 1 MHz, the amplitude was 250 V/cm, and applied for 30 seconds; Then perforated RC electric pulse was added immediately, pulse amplitude was 5 kV/cm, pulse width was 20 s, pulse number was 3, and time interval was 1 second. The fusion solution was left to stand at room temperature for 10 minutes, and then rinsed with a total of 5 mL of PFM solution and taken, and incubated at 37° C. for 30 minutes. Then it was centrifuged at 100 rpm for 8 minutes. The precipitate was taken, and 500 mL of HAT was added, mixed well and dispensed into 96-well culture plate. The number of cells was 1×10⁴ cells/well (there were 1.2*10⁴ cells/well of feeder cells), and put into the CO₂ incubator and cultured at 37° C.

### Example 3 Screening Positive Clones by Indirect ELISA

Positive cell clones were screened by indirect ELISA. The coating concentration for detecting recombinant human TNFR2-His Tag protein (Sino; Cat: 10417-H08H) was determined by square assay. The detection antigen coating buffer was diluted horizontally in a gradient, and 50 μL per well was coated in ELISA plates overnight at 4° C. It was washed three times with PBST, and 200 μL of the blocking solution was added to each well overnight at 4° C. The immunized mouse serum was diluted longitudinally in multiplicity at 50 μL per well, and the same multiplicity of dilution was used as the negative control for normal rat serum, and incubated at 37° C. for 2 h. It was washed three times with PBST, and the working concentration of enzyme-labeled secondary antibody was added at 50 μL per well, and incubated at 37° C. for 1.5 h. after washing with PBST, the TMB color was developed, and the value of OD450 was determined by enzyme-linked detector to determine the optimal coating concentration for the detection of antigen.

The antibody secreted by hybridoma cells was detected by the constructed indirect ELISA. The method was as follows: hybridoma cell culture supernatants were added into ELISA plates pre-coated with recombinant human TNFR2-His Tag protein (Sino; Cat: 10417-H08H) at 50 μL/well. SP2/0 cell supernatants as the negative control and immune polyantibody serum as the positive control were added in water bath at 37° C. for 2 h. It was washed three times with PBST. HRP-labeled goat anti-mouse IgG and IgM antibodies at working concentration were added in the water bath at 37° C. for 1.5 h at 50 μL/well. After washing, TMB was used to develop the color for 10 min, and the OD450 reading was determined by an enzyme marker after display termination. The OD450 reading of the tested wells was more than twice that of the negative control, which was judged as positive.

### Example 4 FACS Method for Further Screening of Positive Clones

Lentiviral vectors carrying human TNFR2, monkey TNFR2 or mouse TNFR2 were transferred into CHOK1 and Flpin-T-Rex293F cell lines. Cells were sorted to establish stable human TNFR2, monkey TNFR2 and mouse TNFR2 expression cell lines, which were CHOK1-hTNFR2, CHOK1-cTNFR2 and Flpin-T-Rex293F-mTNFR2. 1×10⁵ stably expressed cells in 100 μL of FACS buffer were divided into independent tubes, and 100 μL of hybridoma cell culture supernatant was added. The cells were incubated at 4° C. for 30 minutes and then washed twice with excess FACS buffer. The cells were resuspended in 100 μL FACS buffer, and 0.005 g of anti-mouse IgG-PE secondary antibody was added to the sample, incubated for 30 minutes and washed twice with excess FACS buffer. The cells were immobilized in a fixed buffer and subsequently analyzed by flow cytometry. The antibodies that binded specifically to human TNFR2 and were species cross-reactive with monkey and mouse were screened by FACS method.

Monocloning of hybridoma cells was carried out by two rounds of limiting dilution method, and the binding activity of antibodies produced by the subcloned cells to human and monkey TNFR2 proteins was determined by ELISA and FACS assays.

### Example 5 Antibody Preparation

After stable hybridoma cell lines were obtained, monoclonal antibodies were mainly obtained by in vitro culture. The cell lines were expanded into a T75 culture bottle and cultured until the cell coverage was 80-90%. The cell supernatant was discarded, and 30 mL of hybridoma production medium (hybridoma-SFM, Gibco) was added, and cultured at 37° C. and 5% CO₂. Hybridoma production medium (hybridoma-SFM, Gibco) was added after 2-3 days of culture. If the cell survival rate was less than 30%, fresh living cells could be added. After 6-7 days of culture, when the cell survival rate was less than 20%, the culture supernatant was collected after low-speed centrifugation and stored at 4° C. for later use.

The antibody was purified with MabSelect SuRe-LX (GE) affinity chromatography column: \textcircled{1} Column packing: Appropriate amount of the purchased Protein A packing was loaded onto the gravity chromatography column and rinsed to equilibrium with equilibrium buffer (PBS, pH 7.4); \textcircled{2} Loading: The supernatant of the hybridoma cell culture filtered through a 0.22 m membrane was added into the loaded chromatography column, and the flow rate was controlled by 1 drop/second; \textcircled{3} Equilibrium: Equilibrium buffer was used to rinse to equilibrium after loading the sample; \textcircled{4} Elution: Elution buffer (50 mM acetic acid/sodium acetate, pH 3.5) was added to rinse the column and collect the eluate; \textcircled{5} Regeneration: After elution, the column was rinsed with 0.1 M NaOH, which was twice the column volume, and 20% ethanol, which was twice the column volume, and stored at 4° C. Finally, the purity of the antibody was identified by SDS-PAGE and SEC-HPLC, and the concentration of the antibody was determined by UV microspectrophotometer.

### Example 6 FACS Binding Activity of Anti-TNFR2 Antibody to Human TNFR2

CHOK1-hTNFR2 cells were plated at 1×10⁵/well (containing 1% BSA). 100 μL of isotype control and candidate antibodies were added at a concentration of 100 nm and 4-fold diluted in 11 gradients (containing 1% BSA). Cells were incubated at 4° C. for 1 hour and then washed twice with excess FACS buffer. Cells were resuspended in 100 μL of FACS buffer, and 100 μL of goat anti-rat IgG Fc-AF647 (1:500) containing 1% BSA was added, incubated at 4° C. without light for 30 minutes and washed twice with excess FACS buffer. Cells were fixed in fixation buffer and subsequently analyzed by flow cytometry. The FACS method screened for candidate antibodies with high specific binding activity to human TNFR2. The results are shown in Table 1 and FIG. 1, indicating that 11 sequences show equivalent binding ability to hTNFR2 compared with BMK2.

TABLE 1

| | | Maximum average | Parental |
|---|---|---|---|
| Antibody | EC50 (nM) | fluorescence value | cell |
| Antibody 01 | 0.045 | 921 | 92.9 |
| Antibody 02 | 0.067 | 1239 | 65 |
| Antibody 03 | 0.045 | 383 | 64 |
| Antibody 04 | 0.025 | 1322 | 61.3 |
| Antibody 05 | 0.038 | 949 | 64.2 |
| Antibody 06 | 0.077 | 1342 | 74.5 |
| Antibody 07 | 0.050 | 1392 | 64.7 |
| Antibody 08 | 0.081 | 1481 | 71 |
| Antibody 09 | 0.065 | 1370 | 68.9 |
| Antibody 10 | 0.077 | 907 | 64.1 |
| Antibody 11 | 0.493 | 1007 | 76 |
| BMK2 | 0.051 | 1610 | 135 |
| uIgG1 isotype control | >100 | 97 | 134 |

### Example 7 Ligand Blocking Assay

Anti-His antibody (1 μg/mL) was coated at 50 μL/well, incubated at 4° C. overnight and washed 3 times with PBST. 2% BSA was added at 200 μL/well, incubated at room temperature for 1 hour and washed 3 times with PBST. 0.25 μg/mL (containing 2% BSA) of recombinant human TNFR2-His Tag protein (Sino; Cat: 10417-H08H) was added at 50 μL/well, incubated for 1 hour at room temperature and washed 3 times with PBST. Primary antibody (isotype control or candidate antibody) was added at a concentration of 100 nM and diluted 4-fold in 11 gradients with a solution containing 1.6 nM recombinant human TNF-alpha-Biotin protein (Sino; Cat: 10602-HANE-B) and 2% BSA, incubated for 2 hours at room temperature and washed 3 times with PBST. Secondary antibody SA-HRP (1:5000) containing 2% BSA was added at 50 μL/well, incubated for 1 hour at room temperature and washed 3 times with PBST.

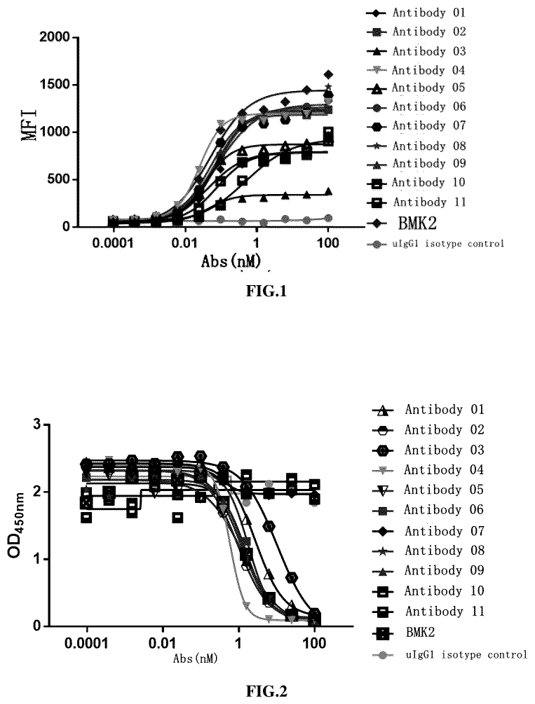

After washing, TMB developed the color for 10 min, and the OD450 reading was determined by the enzyme labeler after the display was terminated. Based on the experimental results, it was judged whether the candidate antibodies had ligand blocking activity. As shown in Table 2 and FIG. 2, 2 sequences show better ligand blocking ability than BMK2; 6 sequences show equivalent ligand blocking ability to BMK2.

TABLE 2

| Antibody | IC50 (nM) | Inhibition rate (%) |
|---|---|---|
| Antibody 01 | 2.72 | 93.2 |
| Antibody 02 | 0.962 | 96.2 |
| Antibody 03 | 10.4 | 91.6 |
| Antibody 04 | 0.584 | 96.0 |
| Antibody 05 | 1.27 | 95.9 |
| Antibody 06 | 1.79 | 96.4 |
| Antibody 07 | >100 | 15.4 |
| Antibody 08 | 1.40 | 96.1 |
| Antibody 09 | 1.18 | 96.1 |
| Antibody 10 | >100 | 12.2 |
| Antibody 11 | >100 | 22.4 |
| BMK2 | 1.54 | 95.3 |
| uIgG1 isotype control | >100 | 11.7 |

### Example 8 Affinity Verification of Anti-TNFR2 Antibodies and Human TNFR2

Equipment: Biacore 8K(GE).
Sensor chip: CM5 chip (GE).

### (1) Immobilization:

Activator preparation: 400 mM EDC and 100 mM NHS (GE) were mixed immediately before use.

The CM5 sensor chip was activated at a flow rate of 10 L/min for 420 s. The channel was then injected with 30 μg/mL of anti-rat Fc IgG in 10 mM NaAc (pH 4.5) at a flow rate of 10 μL/min. The chip was inactivated with 1 M ethanolamine-hydrochloric acid (GE) for 420 s at a flow rate of 10 μL/min.

### (2) Sample Capture:

Candidate antibodies in running buffer 1×HBS-EP+(10 mM HEPES, 150 mM NaCl, 3 mM EDTA, 0.05% Tween 20, pH 7.4) were captured onto Fc2 with anti-rat Fc IgG at a flow rate of 10 μL/min. The 10 nM recombinant human TNFR2-His Tag protein (Sino; Cat: 10417-H08H) and running buffer were sequentially injected into Fc1-Fc2 at a flow rate of 30 min, bound for 180 s, and then dissociated for 3600 s. 10 mM glycine (pH 1.5) was injected as regeneration buffer after each dissociation.

### (3) Regeneration:

The chip was regenerated with 10 mM glycine (pH 1.5).

### (4) Data Analysis:

Result plots for the reference channel Fc1 and the buffer channel were subtracted from the test result plots. The experimental data fit the 1:1 binding model. The molecular weight of 26.6 kDa was used to calculate the molar concentration of recombinant human TNFR2-His Tag protein.

The results are shown in Table 3, and the SPR affinity between the candidate antibodies and human TNFR2 is high, reaching 0.01 nM-1 nM.

TABLE 3
SPR affinity between candidate antibodies and human TNFR2

| Antibody | ka(1/Ms) | kd(1/s) | KD(M) |
|---|---|---|---|
| Antibody 01 | 1.24E+06 | 1.05E−02 | 8.45E−09 |
| Antibody 02 | 3.79E+05 | 1.05E−05 | 2.77E−11 |
| Antibody 03 | 2.22E+05 | 4.13E−03 | 1.87E−08 |
| Antibody 04 | 1.32E+06 | <1.00E−05* | <7.58E−12 |
| Antibody 05 | 2.76E+05 | 2.81E−04 | 1.02E−09 |
| Antibody 06 | 3.18E+05 | 3.33E−04 | 1.05E−09 |
| Antibody 07 | 6.95E+05 | 2.61E−04 | 3.76E−10 |
| Antibody 08 | 3.72E+05 | <1.00E−05* | <2.69E−11 |
| Antibody 09 | 3.73E+05 | 1.37E−05 | 3.66E−11 |
| Antibody 10 | 4.92E+05 | 7.40E−05 | 1.50E−10 |
| Antibody 11 | 1.20E+05 | 1.69E−04 | 1.40E−09 |
| BMK2 | 2.13E+05 | 1.59E−04 | 7.48E−10 |

*Below the lower limit of detection

### Example 9 Anti-TNFR2 Antibody-Mediated Killing Activity Against Treg Cells

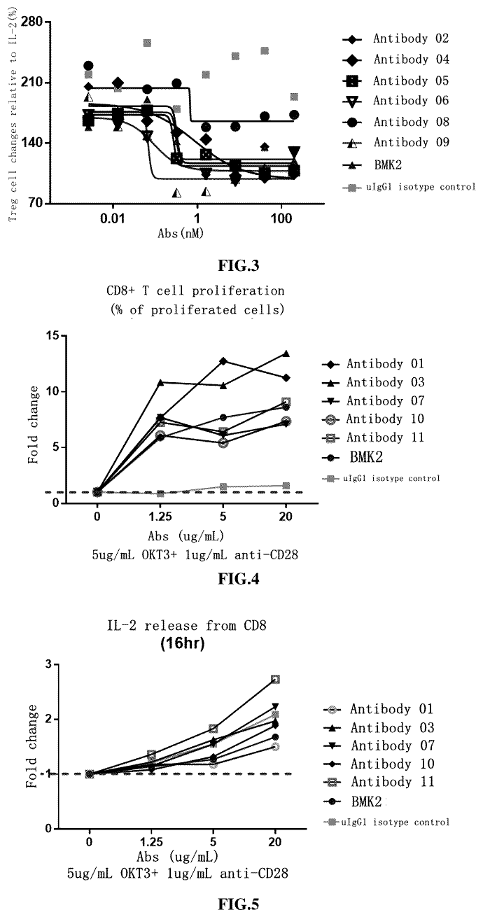

CD4+ T cells were isolated from peripheral blood mononuclear cells (PBMC) of healthy volunteers. 200 nM of candidate antibodies were 5-fold diluted with 9 gradients, and 200 U/mL IL2 and 20 ng/mL recombinant human TNF-alpha were added. CD4+ T cells were added at 2×10⁵/well, and incubated at 37° C. for 72 hours. Staining was performed using PE-anti-CD25 (1:50) and Alexa Fluor 488-anti-Foxp3 (1:50), and analyzed by counting using a BD FACSC anto II flow cytometer. The results are shown in Table 4 and FIG. 3, indicating that all six antibody sequences show very excellent killing activity against Treg cells.

TABLE 4
Anti-TNFR2 antibody-mediated killing activity against Treg cells

| | | Maximum | | Treg cell | |
|---|---|---|---|---|---|
| | | average | | changes | |
| | | fluorescence | | relative to | IL-2 + |
| Antibody | EC50(nM) | value | IC50(nM) | IL-2(%) | hTNFR2L1 |
| Antibody 02 | 0.067 | 1239 | ~0.33 | 96.9 | 187.5 |
| Antibody 04 | 0.025 | 1322 | 0.88 | 100.4 | |
| Antibody 05 | 0.038 | 949 | ~0.27 | 105.1 | |
| Antibody 06 | 0.077 | 1342 | 0.093 | 96.9 | |
| Antibody 09 | 0.065 | 1370 | ~0.068 | 82.7 | |
| Antibody 08 | 0.081 | 1481 | ~0.67 | 158.5 | 187 |
| BMK2 | 0.051 | 1610 | ~0.31 | 108.3 | |
| uIgG1 isotype | >100 | 97.3 | >200 | 173.4 | |
| control | | | | | |

### Example 10 Anti-TNFR2 Antibody-Mediated Proliferative Effects on CD8+ T Cells

Candidate antibodies (containing 5 μg/mL OKT3) were coated at 20 μg/mL, and 4-fold diluted in 3 gradients, and incubated at 4° C. overnight. CD8+ T cells labeled with CFSE were added at 2×10⁵/well, and 1 μg/mL anti-CD28 was added and incubated at 37° C. for 96 hours. Count analysis was performed using BD FACS Canto II flow cytometer. The results are shown in FIG. 4, and all six antibody sequences show very excellent effects on promoting the proliferation of CD8+ T cell.

### Example 11 Anti-TNFR2 Antibody-Mediated Activation of CD8+ T Cells

Candidate antibodies (containing 5 μg/mL OKT3) were coated at 20 μg/mL, and 4-fold diluted in 3 gradients, and incubated at 4° C. overnight. CD8+ T cells were added at 2×10⁵/well, and 1 μg/mL anti-CD28 was added and incubated at 37° C. for 16 hours. The release of IFNγ and IL-2 was detected by ELISA, respectively. The results are shown in FIG. 5, and all six antibody sequences show very excellent effects on promoting the activation of CD8+ T cell.

### Example 12 Sequencing of Monoclonal Antibodies

Three candidate antibodies were selected for sequencing and the amino acid/nucleotide sequences of antibody 04, antibody 10 and antibody 11 were finally obtained. The correspondence between the light chain variable region sequences and the heavy chain variable region sequences and the sequence list is shown in Table 5.

TABLE 5
Correspondence between number and sequence

| | |
|---|---|
| Antibody 04 VH | SEQ ID NO: 01 |
| Antibody 04 VL | SEQ ID NO: 05 |
| Antibody 10 VH | SEQ ID NO: 09 |
| Antibody 10 VL | SEQ ID NO: 13 |
| Antibody 11 VH | SEQ ID NO: 17 |
| Antibody 11 VL | SEQ ID NO: 21 |

### Example 13 Preparation of Recombinant Expression of Chimeric Antibodies

DNA fragments of gene synthesis light chain variable region and human IgG1 kappa light chain constant region (SEQ ID NO: 69) were light chain DNA, and DNA fragments of gene synthesis heavy chain variable region and human IgG1 heavy chain constant region (SEQ ID NO: 70) were heavy chain DNA. Mammalian cell expression plasmid was constructed by inserting into pcDNA3.1-G418 vector using molecular cloning technique. The pcDNA3.1-G418 vector contained promoter CMV Promoter, eukaryotic screening marker G418 tag and prokaryotic screening tag Ampicillin. Nucleotide sequences of antibody 04-xIgG1K, antibody 10-xIgG1K, antibody 11-xIgG1K antibody expression light chain and heavy chain were obtained by gene synthesis, and the vector and target fragment were double-digested with HindIII and XhoI, recovered and then enzymatically ligated by DNA ligase and transformed into E. coli competent cell DH5a. Positive clones were picked out, plasmid extracted and enzymatically verified, and recombinant plasmids containing antibody 04-xIgG1K, antibody 10-xIgG1K, antibody 11-xIgG1K heavy chain and light chain were obtained.

Recombinant plasmids containing each of the above target genes were transformed into E. coli competent cells DH5a according to the method described in the Guidelines for Molecular Cloning Experiments (2002, Science Press), and the transformed bacteria were coated and cultured on the LB plate containing 100 μg/mL of ampicillin, and the plasmid were selected and cloned into the liquid LB medium for culture. The bacteria was shaken at 260 rpm for 14 hours. The plasmids were extracted by the endotoxin-free plasmid macroabsorbent kit, and solubilized in sterile water, and the concentration was determined by the nucleic acid protein quantifier.

ExpiCHO was cultured to a cell density of 6×10⁶ cells/mL at 37° C., 8% CO₂, and 100 rpm. Recombinant plasmids containing antibody 04-xIgG1K, antibody 10-xIgG1K, and antibody 11-xIgG1K heavy chain and light chain were transfected into the above cells by liposomes at a concentration of 1 μg/mL, and the concentration of liposomes was determined according to the ExpiCHO™ Expression System kit, and cultured at 32° C., 5% CO₂, and 100 rpm for 7-10 days. Supplement was given once after 18-22 h of transfection and between day 5-8, respectively. The culture product was centrifuged at 4000 rpm, and filtered through a 0.22 m filter membrane and the supernatant of the medium was collected, and the antibodies were purified by using Protein A.

### Example 14 Evaluation of In Vivo Anti-Tumor Efficacy of Chimeric Antibodies

The in vivo anti-tumor efficacy of antibody 04-xIgG1K, antibody 10-xIgG1K, and antibody 11-xIgG1K was evaluated in the humanized mouse model of tumor C57BL/6-hTNFR2 transplanted from mouse colon cancer cell line MC38.

The mouse colon cancer cell MC38 were cultured in a single layer in vitro in RPMI1640 medium supplemented with 10% fetal bovine serum and 2 mM glutamine at 37° C. and 5% CO₂. It was routinely digested and passaged by trypsin-EDTA twice a week. When the cell saturation was 80-90%, cells were collected, counted and inoculated. 0.1 mL (5×10⁵ cells) of MC38 cells were subcutaneously inoculated into the right back of each mouse. When the average tumor volume reached 64 mm³, administration was started in groups.

Antibody 04-xIgG1K, antibody 10-xIgG1K, and antibody 11-xIgG1K were administered intraperitoneally to the tumor-bearing mice twice weekly at 2.0 mg/kg each time, and the same dose of human IgG was administered as a control for a total of 5 times.

The health and death of the animals were monitored every day. Routine examinations include observation of the effects of tumor growth and drug treatment on the animals' daily behavior performance such as behavioral activities, food and water intake (visual observation only), weight changes (weight measurement three times a week), physical signs or other abnormal conditions. The number of deaths and side effect of animals in each group were recorded.

The experimental index was to investigate whether the tumor growth was inhibited, delayed or cured. The tumor diameter was measured with a vernier caliper three times a week. The calculation formula of tumor volume was: V=0.5a×b², wherein a and b represented the long and short diameter of the tumor, respectively. The tumor suppressive efficacy of the compound was evaluated by TGI (%) or the relative tumor proliferation rate T/C (%). TGI (%) reflected the tumor growth inhibition rate. Calculation of TGI (%) was: TGI (%)=[(1−(Average tumor volume at the end of a certain treatment group-Average tumor volume at the beginning of the treatment group))/(Average tumor volume at the end of treatment in the solvent control group-Average tumor volume at the start of treatment in the solvent control group)]×100%. Calculation of the relative tumor proliferation rate T/C (%) was: T/C %=T_{RTV}/C_{RTV}×100% (T_{RTV}: treatment group RTV; C_{RTV}: negative control group RTV). Relative tumor volume (RTV) was calculated based on the results of tumor measurement. The calculation formula was RTV=Vₜ/V₀, where V₀ was the average tumor volume measured during group administration (that was, d0), and Vt was the average tumor volume during a certain measurement. T_{RTV} and C_{RTV} were taken from the same day.

Tumor-bearing mice of mouse colon cancer cell line MC38 transplanted tumor model were given human IgG control, antibody 04-xIgG1K, antibody 10-xIgG1K and antibody 11-xIgG1K, respectively. The tumor growth curves are shown in FIG. 6, where the horizontal coordinates indicate the number of days since the start of the treatment, and the vertical coordinates indicate the tumor volume. As shown in FIG. 6, antibody 04-xIgG1K, antibody 10-xIgG1K, antibody 11-xIgG1K, and BMK6 have equivalent or better in vivo anti-tumor efficacy, indicating that antibody 11-xIgG1K has significantly better in vivo tumor efficacy than BMK6.

### Example 15 Humanization of Antibodies

The VH and VL sequences were compared with libraries of known human ethnographic lineage sequences on the NCBI website (https://www.ncbi.nlm.nih.gov/igblast/). The databases used were the IMGT human VH gene and the IMGT human VLK gene. For antibody 04 VH, human germline IGVH1-2 was selected as the acceptor sequence, and the human light chain IGKJ4 (allele 1) joining region (J gene) was selected from the human joining region sequences compiled at IMGT®, the international ImMunoGeneTics information System® http://www.imgt.org. For antibody 04 VL, human germline IGKV1-16 was selected as the acceptor sequence, and the human light chain IGKJ2 (allele 1) joining region (J gene) was selected from the human joining region sequences compiled at IMGT®, the international ImMunoGeneTics information System® http://www.imgt.org. For antibody 10 VH, human germline IGVH3-15 was selected as the acceptor sequence, and the human light chain IGKJ4 (allele 1) joining region (J gene) was selected from the human joining region sequences compiled at IMGT®, the international ImMunoGeneTics information System® http://www.imgt.org. For antibody 10 VL, human germline IGKV1-27 was selected as the acceptor sequence, and the human light chain IGKJ2 (allele 1) joining region (J gene) was selected from the human joining region sequences compiled at IMGT®, the international ImMunoGeneTics information System® http://www.imgt.org. For antibody 11 VH, human germline IGVH1-69 was selected as the acceptor sequence, and the human light chain IGKJ4 (allele 1) joining region (J gene) was selected from the human joining region sequences compiled at IMGT®, the international ImMunoGeneTics information System® http://www.imgt.org. For antibody 11 VL, human germline IGKV3D-15 was selected as the acceptor sequence, and the human light chain IGKJ2 (allele 1) joining region (J gene) was selected from the human joining region sequences compiled at IMGT®, the international ImMunoGeneTics information System® http://www.imgt.org. The CDRs were determined according to the AbM definition, so that the human germline frame positions were changed to the corresponding parental mouse sequences to optimize the binding of the humanized antibodies. By the above method, the following multiple humanized antibodies were obtained.

| Antibody | Sequence number |
|---|---|
| Antibody 12 | VH: SEQ ID NO: 25; VL: SEQ ID NO: 26 |
| Antibody 13 | VH: SEQ ID NO: 31; VL: SEQ ID NO: 32 |
| Antibody 14 | VH: SEQ ID NO: 34; VL: SEQ ID NO: 35 |
| Antibody 15 | VH: SEQ ID NO: 37; VL: SEQ ID NO: 38 |
| Antibody 16 | VH: SEQ ID NO: 41; VL: SEQ ID NO: 42 |
| Antibody 17 | VH: SEQ ID NO: 43; VL: SEQ ID NO: 44 |
| Antibody 18 | VH: SEQ ID NO: 29; VL: SEQ ID NO: 30 |
| Antibody 19 | VH: SEQ ID NO: 39; VL: SEQ ID NO: 40 |
| Antibody 20 | VH: SEQ ID NO: 46; VL: SEQ ID NO: 47 |
| Antibody 21 | VH: SEQ ID NO: 49; VL: SEQ ID NO: 50 |
| Antibody 22 | VH: SEQ ID NO: 51; VL: SEQ ID NO: 52 |
| Antibody 23 | VH: SEQ ID NO: 53; VL: SEQ ID NO: 54 |
| Antibody 24 | VH: SEQ ID NO: 56; VL: SEQ ID NO: 57 |
| Antibody 25 | VH: SEQ ID NO: 59; VL: SEQ ID NO: 60 |
| Antibody 26 | VH: SEQ ID NO: 62; VL: SEQ ID NO: 63 |
| Antibody 27 | VH: SEQ ID NO: 65; VL: SEQ ID NO: 66 |
| Antibody 28 | VH: SEQ ID NO: 67; VL: SEQ ID NO: 68 |

### Example 16 Assay of Binding Activity of the Humanized Antibodies

(1) Coating: Human-TNFR2-His was diluted to 0.5 μg/mL with coating solution (1×PBS, pH 7.4), and coated into a 96-well ELISA plate at 100 μL/well at 4° C. overnight. The coating solution was poured off, and the plate was washed with 1×PBST at 300 μL per well, washed with a plate cleaning instrument for 4 times, and patted dry on sheet paper.
(2) Blocking: it was blocked with 3% non-fat dry milk at 300 μL/well, and incubated at 37° C. for 1 h. The blocking solution was poured off, washed by a plate cleaning instrument for 4 times, and patted dry on sheet paper.
(3) Sample dilution: the reference product and the test product were diluted with 3% non-fat dry milk to 10 μg/mL, and this was used as the initial concentration for 3-fold dilution with a total of 11 gradients of dilution, and another one blank well was set up, and only diluent was added. It was incubated at 100 μL/well at 37° C. for 1 h. Liquid in the wells was discarded, washed by a plate cleaning instrument for 4 times, and patted dry on the sheet paper.
(4) Addition of enzyme-labeled secondary antibody: Peroxidase-conjugated AffiniPure F(ab′)2 Fragment Goat Anti-Human IgG was diluted with 3% non-fat dry milk at 1:20,000. It was incubated at 100 μL/well at 37° C. for 1 h. It was washed by a plate cleaning instrument for 6 times and patted dry on sheet paper.
(5) Color development: TMB color development solution was added at 100 μL/well and wrapped with aluminium foil, and the color was developed at 37° C. without light for 8 min.
(6) Termination of color development: terminal solution 1 M HCl was added to terminate the color development reaction at 100 μL/well.
(7) Reading at 450 nm on the enzyme labeler.
(8) The humanized antibodies have equivalent or better binding activity compared with the corresponding chimeric antibody, indicating that the antibodies can maintain high binding activity after humanization.

TABLE 6

| Antibody | EC50 (μg/mL) |
|---|---|
| Antibody 04-xIgG1K(Reference) | 0.005815 |
| Antibody 12 | 0.01348 |
| Antibody 13 | 0.004955 |
| Antibody 14 | 0.00375 |
| Antibody 15 | 0.003197 |
| Antibody 16 | 0.02026 |
| Antibody 17 | 0.01488 |
| Antibody 10-xIgG1K(Reference) | 0.01429 |
| Antibody 21 | 0.01469 |
| Antibody 22 | 0.01373 |

### Example 17 Affinity Assay of the Humanized Antibodies

Equipment: OCTET Red96e (Fortebio).
Sensor: AHC.

### (1) Experimental Setup:

Sensor preparation: the AHC sensor was soaked in 0.02% PBST (0.02% Tween 20, pH 7.4, 1*PBS) as buffer for 600 s prior to use to remove the sucrose covering the sensor surface.

### (2) Immobilization and Capture:

After equilibration of the AHC sensor with 0.02% PBST (0.02% Tween 20, pH 7.4, 1*PBS) as buffer for 60 s, the TNFR2 antibody in the sample plate was immobilized for 300 s, and second equilibration was performed for 180 s. 100 nM of human W3785-hProl.ECD.His (Human-TNFR2-His) protein was bound with the TNFR2 antibody for 300 s and then dissociated for 600 s. After dissociation, 10 mM glycine (pH 2.0) was used as regeneration buffer to regenerate for 30 s.

### (3) Regeneration:

The sensor was regenerated with 10 mM glycine (pH 2.0).

### (4) Data Analysis:

As shown in Table 7, the humanized antibodies have equivalent or better binding activity compared with the corresponding antibodies, indicating that the humanized antibodies can maintain a high affinity.

TABLE 7
Affinity assay results

| | Affinity KD | Affinity Kon | Affinity Kdis | Affinity KD |
|---|---|---|---|---|
| | (single | (multiple | (multiple | (multiple |
| Antibody | concentration) | concentrations) | concentrations) | concentrations) |
| Antibody 04- | 1.48E−10 | 1.1E+06 | 6.65E−05 | 6.06E−11 |
| xIgG1K | | | | |
| (Reference) | | | | |
| Antibody 12 | Untested | 1.21E+06 | 1.02E−04 | 8.63E−11 |
| Antibody 13 | Untested | 1.15E+06 | 1.34E−04 | 1.17E−10 |
| Antibody 14 | Untested | 9.31E+05 | 2.82E−03 | 3.03E−09 |
| Antibody 15 | Untested | 2.33E+04 | 1.76E−05 | 2.06E−09 |
| Antibody 16 | Untested | 1.15E+06 | 1.34E−04 | 1.17E−10 |
| Antibody 17 | Untested | 9.33E+05 | 1.24E−04 | 1.33E−10 |
| Antibody 18 | Untested | 9.74E+05 | 6.22E−05 | 6.38E−11 |
| Antibody 19 | Untested | 8.62E+05 | 1.27E−03 | 1.48E−09 |
| Antibody 20 | Untested | 6.58E+05 | 1.32E−04 | 2.01E−10 |
| Antibody 10- | 2.40E−10 | 8.13E+05 | 8.10E−05 | 9.96E−11 |
| xIgG1K | | | | |
| (Reference) | | | | |
| Antibody 21 | Untested | 8.21E+05 | 1.45E−04 | 1.77E−10 |
| Antibody 22 | Untested | 5.0E+05 | 1.65E−04 | 3.3E−10 |
| Antibody 11- | 3.96E−10 | 5.42E+05 | 1.713E−04 | 3.168E−10 |
| xIgG1K | | | | |
| (Reference) | | | | |
| Antibody 23 | Untested | 4.84E+05 | 3.71E−04 | 7.66E−10 |
| Antibody 24 | Untested | 7.18E+05 | 1.09E−04 | 1.52E−10 |
| Antibody 25 | Untested | 1.39E+06 | 1.93E−03 | 1.39E−09 |
| Antibody 26 | Untested | 2.56E+05 | 1.41E−05 | 5.51E−11 |
| Antibody 27 | Untested | 1.44E+06 | 7.70E−04 | 5.37E−10 |
| Antibody 28 | Untested | 2.25E+05 | <1.0E−07 | <1.0E−12 |

### Example 18 In Vivo Efficacy of the Drug in the C57BL/6hTNFR2 Mouse Model of Transplanted Tumor of the Mouse Colon Cancer Cell Line MC38

Mouse colon cancer MC38 cells were cultured in single layer in vitro with DMEM medium supplemented with 10% fetal bovine serum, 2 mM glutamine at 37° C. and 5% CO₂. Passage was performed by routine digestion twice a week with trypsin-EDTA. When the cell saturation was 80-90%, the cells were collected, counted, and inoculated.

0.1 mL (5×10⁵ cells) of MC38 cells were subcutaneously inoculated into the right back of each mouse. The drug was administered in groups when the average tumor volume reached 54 mm³. The experimental index was to examine whether tumor growth was inhibited, delayed or cured. Tumor diameter was measured three times a week. The calculation formula for tumor volume is V=0.5a×b², where a and b represent the long and short diameters of the tumor, respectively. Tumor inhibition efficacy was evaluated by TGI (%). TGI (%) reflects tumor growth inhibition rate. Calculation of TGI (%): TGI (%)=[(1−(mean tumor volume at the end of administration of a treatment group−mean tumor volume at the beginning of administration of the treatment group))/(mean tumor volume at the end of treatment of the solvent control group−mean tumor volume at the beginning of treatment of the solvent control group)]×100%.

TABLE 8
Grouping of animals for in vivo potency experiments and administration regimens

| | Number | Compound | Dosage | Route of | Frequency of |
|---|---|---|---|---|---|
| Group | of mice | treatment | (mg/kg) | administration | administration |
| 1 | 4 | Physiological | — | Intraperitoneal | 2 times per week for |
| | | saline | | injection | a total of 10 times |
| 2 | 4 | BMK6 | 2 | Intraperitoneal | 2 times per week for |
| | | | | injection | a total of 10 times |
| 3 | 4 | Antibody 24 | 2 | Intraperitoneal | 2 times per week for |
| | | | | injection | a total of 10 times |

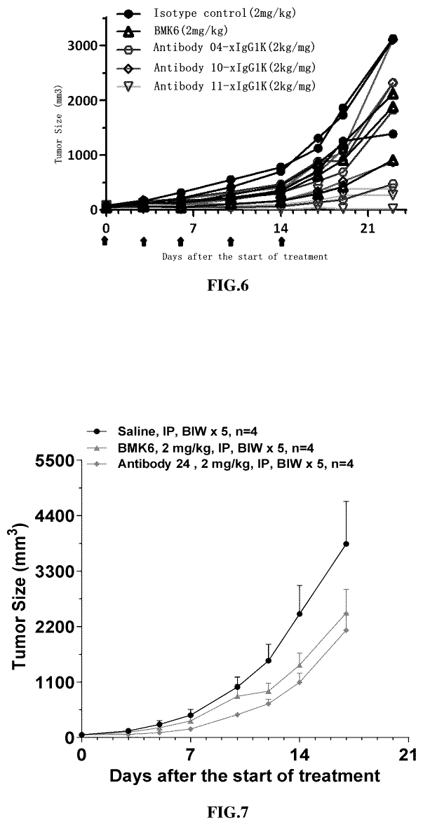

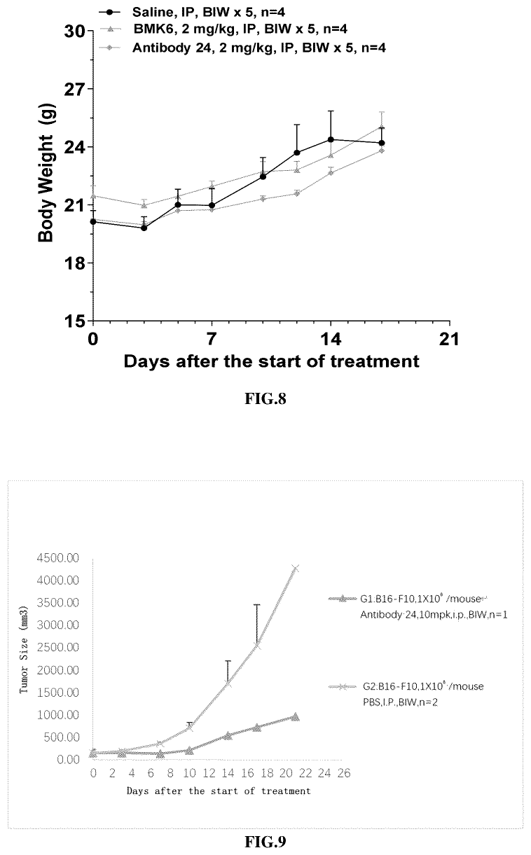

The results are shown in FIG. 7, where the horizontal coordinate indicates the number of days after group administration and the vertical coordinate indicates the tumor volume. After 6 days of cell inoculation, 54 mm³ was reached for compartmentalization and administration, and after 14 days of administration, the average tumor-bearing volume of mice in the physiological saline control group reached 2452±563 mm³; while the tumor-bearing volume of mice in the constructed antibody treatment group was 1093±184 mm³, and the tumor growth was significantly inhibited (TGI was 56.65%), and the body weight of mice in both the physiological saline control group and the constructed antibody treatment group grew slowly (e.g., FIG. 8), which indicated that the constructed antibody shows good anti-tumor activity and has a good safety.

### Example 19 In Vivo Efficacy of the Drug in the C57BL/6hTNFR2 Mouse Model of Transplanted Tumor of the Mouse Melanoma Cell Line B16-F10

1×10⁶ mouse melanoma B16-F10 cells were injected subcutaneously into the right back of female nude mice to establish a subcutaneously transplanted tumor model. The drug was administered in groups when the average tumor volume reached 150 mm³. 10 mpk constructed antibody, 10 mpk isotype control, or an equal volume of PBS was used for the treatment of intraperitoneal injection, which were administered every 3 days and twice a week. The experimental index was to investigate whether the tumor growth was inhibited, delayed or cured. The tumor diameter was measured three times a week. The calculation formula of tumor volume was: V=0.5a×b², wherein a and b represented the long and short diameter of the tumor, respectively. The tumor suppressive efficacy was evaluated by TGI (%). TGI (%) reflected the tumor growth inhibition rate. Calculation of TGI (%) was: TGI (%)=[(1−(Average tumor volume at the end of a certain treatment group−Average tumor volume at the beginning of the treatment group))/(Average tumor volume at the end of treatment in the solvent control group−Average tumor volume at the start of treatment in the solvent control group)]×100%.

The results are shown in FIG. 9, where the horizontal coordinate indicates the number of days after group administration and the vertical coordinate indicates the tumor volume. After 8 days of cell inoculation, 150 mm³ was reached for compartmentalization and administration, and after 21 days of administration, the average tumor-bearing volume of mice in the PBS control and isotype control treatment groups reached 4273 mm³; while the tumor-bearing volume of mice in the constructed antibody treatment group was only 967 mm³, and the tumor growth was significantly inhibited (TGI was 80.3%), and the constructed antibody shows good anti-tumor activity.

1. An antibody or antigen-binding fragment thereof capable of specifically binding to human tumor necrosis factor receptor 2 (TNFR2), wherein the antibody or antigen-binding fragment comprises heavy chains and light chains, and the heavy chain variable region comprises the heavy chain complementary determining regions CDR1, CDR2 and CDR3, and the light chain variable region comprises the light chain complementary determining regions CDR1, CDR2 and CDR3, wherein,
(01) CDR1, CDR2 and CDR3 of the heavy chain variable region consist of the amino acid sequences SEQ ID NO: 18, SEQ ID NO: 58 and SEQ ID NO: 20, respectively, and CDR1, CDR2 and CDR3 of the light chain variable region consist of SEQ ID NO: 22, SEQ ID NO: 23 and SEQ ID NO: 24, respectively;
(02) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 6, SEQ ID NO: 7 and SEQ ID NO: 8, respectively;
(03) CDR1, CDR2 and CDR3 of the heavy chain variable region consist of the amino acid sequences SEQ ID NO: 10, SEQ ID NO: 11 and SEQ ID NO: 12, respectively, and CDR1, CDR2 and CDR3 of the light chain variable region consist of SEQ ID NO: 14, SEQ ID NO: 15 and SEQ ID NO: 16, respectively;
(04) CDR1, CDR2 and CDR3 of the heavy chain variable region consist of the amino acid sequences SEQ ID NO: 18, SEQ ID NO: 19 and SEQ ID NO: 20, respectively, and CDR1, CDR2 and CDR3 of the light chain variable region consist of SEQ ID NO: 22, SEQ ID NO: 23 and SEQ ID NO: 24, respectively;
(05) CDR1, CDR2 and CDR3 of the heavy chain variable region consist of the amino acid sequences SEQ ID NO: 10, SEQ ID NO: 11 and SEQ ID NO: 12, respectively, and CDR1, CDR2 and CDR3 of the light chain variable region consist of SEQ ID NO: 27, SEQ ID NO: 15 and SEQ ID NO: 16, respectively;
(06) CDR1, CDR2 and CDR3 of the heavy chain variable region consist of the amino acid sequences SEQ ID NO: 18, SEQ ID NO: 55 and SEQ ID NO: 20, respectively, and CDR1, CDR2 and CDR3 of the light chain variable region consist of SEQ ID NO: 22, SEQ ID NO: 23 and SEQ ID NO: 24, respectively;
(07) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 28, SEQ ID NO: 7 and SEQ ID NO: 8, respectively;
(08) CDR1, CDR2 and CDR3 of the heavy chain variable region consist of the amino acid sequences SEQ ID NO: 2, SEQ ID NO: 3 and SEQ ID NO: 4, respectively, and CDR1, CDR2 and CDR3 of the light chain variable region consist of SEQ ID NO: 33, SEQ ID NO: 7 and SEQ ID NO: 8, respectively;
(09) CDR1, CDR2 and CDR3 of the heavy chain variable region consist of the amino acid sequences SEQ ID NO: 2, SEQ ID NO: 3 and SEQ ID NO: 36, respectively, and CDR1, CDR2 and CDR3 of the light chain variable region consist of SEQ ID NO: 6, SEQ ID NO: 7 and SEQ ID NO: 8, respectively;
(10) CDR1, CDR2 and CDR3 of the heavy chain variable region consist of the amino acid sequences SEQ ID NO: 18, SEQ ID NO: 55 and SEQ ID NO: 61, respectively, and CDR1, CDR2 and CDR3 of the light chain variable region consist of SEQ ID NO: 22, SEQ ID NO: 23 and SEQ ID NO: 24, respectively;
(11) CDR1, CDR2 and CDR3 of the heavy chain variable region consist of the amino acid sequences SEQ ID NO: 18, SEQ ID NO: 55 and SEQ ID NO: 64, respectively, and CDR1, CDR2 and CDR3 of the light chain variable region consist of SEQ ID NO: 22, SEQ ID NO: 23 and SEQ ID NO: 24, respectively;
(12) CDR1, CDR2 and CDR3 of the heavy chain variable region consist of the amino acid sequences SEQ ID NO: 18, SEQ ID NO: 58 and SEQ ID NO: 61, respectively, and CDR1, CDR2 and CDR3 of the light chain variable region consist of SEQ ID NO: 22, SEQ ID NO: 23 and SEQ ID NO: 24, respectively;
(13) CDR1, CDR2 and CDR3 of the heavy chain variable region consist of the amino acid sequences SEQ ID NO: 2, SEQ ID NO: 3 and SEQ ID NO: 36, respectively, and CDR1, CDR2 and CDR3 of the light chain variable region consist of SEQ ID NO: 28, SEQ ID NO: 7 and SEQ ID NO: 8, respectively;
(14) CDR1, CDR2 and CDR3 of the heavy chain variable region consist of the amino acid sequences SEQ ID NO: 2, SEQ ID NO: 3 and SEQ ID NO: 45, respectively, and CDR1, CDR2 and CDR3 of the light chain variable region consist of SEQ ID NO: 6, SEQ ID NO: 7 and SEQ ID NO: 8, respectively;
(15) CDR1, CDR2 and CDR3 of the heavy chain variable region consist of the amino acid sequences SEQ ID NO: 2, SEQ ID NO: 3 and SEQ ID NO: 45, respectively, and CDR1, CDR2 and CDR3 of the light chain variable region consist of SEQ ID NO: 28, SEQ ID NO: 7 and SEQ ID NO: 48, respectively; or
(16) CDR1, CDR2 and CDR3 of the heavy chain variable region consist of the amino acid sequences SEQ ID NO: 18, SEQ ID NO: 58 and SEQ ID NO: 64, respectively, and CDR1, CDR2 and CDR3 of the light chain variable region consist of SEQ ID NO: 22, SEQ ID NO: 23 and SEQ ID NO: 24, respectively.

2. The antibody or antigen-binding fragment thereof of claim 1, wherein:
(01) the heavy chain variable region has the amino acid sequence shown in SEQ ID NO: 56, or a sequence having at least 80%, 85%, 90%, 91%, 92%, 93%, 94%, 95%, 96%, 97%, 98%, 99% or more identity to the amino acid sequence shown in SEQ ID NO: 56, or an amino acid sequence having one or more conserved amino acid mutations in comparison to the amino acid sequence of SEQ ID NO: 56, and
the light chain variable region has the amino acid sequence shown in SEQ ID NO: 57, or a sequence having at least 80%, 85%, 90%, 91%, 92%, 93%, 94%, 95%, 96%, 97%, 98%, 99% or more identity to the amino acid sequence shown in SEQ ID NO: 57, or an amino acid sequence having one or more conserved amino acid mutations in comparison to the amino acid sequence of SEQ ID NO: 56,
(02) the heavy chain variable region has the amino acid sequence shown in SEQ ID NO: 1, or a sequence having at least 80%, 85%, 90%, 91%, 92%, 93%, 94%, 95%, 96%, 97%, 98%, 99% or more identity to the amino acid sequence shown in SEQ ID NO: 1, or an amino acid sequence having one or more conserved amino acid mutations in comparison to the amino acid sequence of SEQ ID NO: 1, and
the light chain variable region has the amino acid sequence shown in SEQ ID NO: 5, or a sequence having at least 80%, 85%, 90%, 91%, 92%, 93%, 94%, 95%, 96%, 97%, 98%, 99% or more identity to the amino acid sequence shown in SEQ ID NO: 5, or an amino acid sequence having one or more conserved amino acid mutations in comparison to the amino acid sequence of SEQ ID NO: 5,
(03) the heavy chain variable region has the amino acid sequence shown in SEQ ID NO: 9, or a sequence having at least 80%, 85%, 90%, 91%, 92%, 93%, 94%, 95%, 96%, 97%, 98%, 99% or more identity to the amino acid sequence shown in SEQ ID NO: 9, or an amino acid sequence having one or more conserved amino acid mutations in comparison to the amino acid sequence of SEQ ID NO: 9, and
the light chain variable region has the amino acid sequence shown in SEQ ID NO: 13, or a sequence having at least 80%, 85%, 90%, 91%, 92%, 93%, 94%, 95%, 96%, 97%, 98%, 99% or more identity to the amino acid sequence shown in SEQ ID NO: 13, or an amino acid sequence having one or more conserved amino acid mutations in comparison to the amino acid sequence of SEQ ID NO: 13,
(04) the heavy chain variable region has the amino acid sequence shown in SEQ ID NO: 17, or a sequence having at least 80%, 85%, 90%, 91%, 92%, 93%, 94%, 95%, 96%, 97%, 98%, 99% or more identity to the amino acid sequence shown in SEQ ID NO: 17, or an amino acid sequence having one or more conserved amino acid mutations in comparison to the amino acid sequence of SEQ ID NO: 17, and
the light chain variable region has the amino acid sequence shown in SEQ ID NO: 21, or a sequence having at least 80%, 85%, 90%, 91%, 92%, 93%, 94%, 95%, 96%, 97%, 98%, 99% or more identity to the amino acid sequence shown in SEQ ID NO: 21, or an amino acid sequence having one or more conserved amino acid mutations in comparison to the amino acid sequence of SEQ ID NO: 21,
(05) the heavy chain variable region has the amino acid sequence shown in SEQ ID NO: 49, or a sequence having at least 80%, 85%, 90%, 91%, 92%, 93%, 94%, 95%, 96%, 97%, 98%, 99% or more identity to the amino acid sequence shown in SEQ ID NO: 49, or an amino acid sequence having one or more conserved amino acid mutations in comparison to the amino acid sequence of SEQ ID NO: 49, and
the light chain variable region has the amino acid sequence shown in SEQ ID NO: 50, or a sequence having at least 80%, 85%, 90%, 91%, 92%, 93%, 94%, 95%, 96%, 97%, 98%, 99% or more identity to the amino acid sequence shown in SEQ ID NO: 50, or an amino acid sequence having one or more conserved amino acid mutations in comparison to the amino acid sequence of SEQ ID NO: 50,
(06) the heavy chain variable region has the amino acid sequence shown in SEQ ID NO: 51, or a sequence having at least 80%, 85%, 90%, 91%, 92%, 93%, 94%, 95%, 96%, 97%, 98%, 99% or more identity to the amino acid sequence shown in SEQ ID NO: 51, or an amino acid sequence having one or more conserved amino acid mutations in comparison to the amino acid sequence of SEQ ID NO: 51, and
the light chain variable region has the amino acid sequence shown in SEQ ID NO: 52, or a sequence having at least 80%, 85%, 90%, 91%, 92%, 93%, 94%, 95%, 96%, 97%, 98%, 99% or more identity to the amino acid sequence shown in SEQ ID NO: 52, or an amino acid sequence having one or more conserved amino acid mutations in comparison to the amino acid sequence of SEQ ID NO: 52,
(07) the heavy chain variable region has the amino acid sequence shown in SEQ ID NO: 53, or a sequence having at least 80%, 85%, 90%, 91%, 92%, 93%, 94%, 95%, 96%, 97%, 98%, 99% or more identity to the amino acid sequence shown in SEQ ID NO: 53, or an amino acid sequence having one or more conserved amino acid mutations in comparison to the amino acid sequence of SEQ ID NO: 53, and
the light chain variable region has the amino acid sequence shown in SEQ ID NO: 54, or a sequence having at least 80%, 85%, 90%, 91%, 92%, 93%, 94%, 95%, 96%, 97%, 98%, 99% or more identity to the amino acid sequence shown in SEQ ID NO: 54, or an amino acid sequence having one or more conserved amino acid mutations in comparison to the amino acid sequence of SEQ ID NO: 54,
(08) the heavy chain variable region has the amino acid sequence shown in SEQ ID NO: 25, or a sequence having at least 80%, 85%, 90%, 91%, 92%, 93%, 94%, 95%, 96%, 97%, 98%, 99% or more identity to the amino acid sequence shown in SEQ ID NO: 25, or an amino acid sequence having one or more conserved amino acid mutations in comparison to the amino acid sequence of SEQ ID NO: 25, and
the light chain variable region has the amino acid sequence shown in SEQ ID NO: 26, or a sequence having at least 80%, 85%, 90%, 91%, 92%, 93%, 94%, 95%, 96%, 97%, 98%, 99% or more identity to the amino acid sequence shown in SEQ ID NO: 26, or an amino acid sequence having one or more conserved amino acid mutations in comparison to the amino acid sequence of SEQ ID NO: 26,
(09) the heavy chain variable region has the amino acid sequence shown in SEQ ID NO: 29, or a sequence having at least 80%, 85%, 90%, 91%, 92%, 93%, 94%, 95%, 96%, 97%, 98%, 99% or more identity to the amino acid sequence shown in SEQ ID NO: 29, or an amino acid sequence having one or more conserved amino acid mutations in comparison to the amino acid sequence of SEQ ID NO: 29, and
the light chain variable region has the amino acid sequence shown in SEQ ID NO: 30, or a sequence having at least 80%, 85%, 90%, 91%, 92%, 93%, 94%, 95%, 96%, 97%, 98%, 99% or more identity to the amino acid sequence shown in SEQ ID NO: 30, or an amino acid sequence having one or more conserved amino acid mutations in comparison to the amino acid sequence of SEQ ID NO: 30,
(10) the heavy chain variable region has the amino acid sequence shown in SEQ ID NO: 31, or a sequence having at least 80%, 85%, 90%, 91%, 92%, 93%, 94%, 95%, 96%, 97%, 98%, 99% or more identity to the amino acid sequence shown in SEQ ID NO: 31, or an amino acid sequence having one or more conserved amino acid mutations in comparison to the amino acid sequence of SEQ ID NO: 31, and
the light chain variable region has the amino acid sequence shown in SEQ ID NO: 32, or a sequence having at least 80%, 85%, 90%, 91%, 92%, 93%, 94%, 95%, 96%, 97%, 98%, 99% or more identity to the amino acid sequence shown in SEQ ID NO: 32, or an amino acid sequence having one or more conserved amino acid mutations in comparison to the amino acid sequence of SEQ ID NO: 32,
(11) the heavy chain variable region has the amino acid sequence shown in SEQ ID NO: 34, or a sequence having at least 80%, 85%, 90%, 91%, 92%, 93%, 94%, 95%, 96%, 97%, 98%, 99% or more identity to the amino acid sequence shown in SEQ ID NO: 34, or an amino acid sequence having one or more conserved amino acid mutations in comparison to the amino acid sequence of SEQ ID NO: 34, and
the light chain variable region has the amino acid sequence shown in SEQ ID NO: 35, or a sequence having at least 80%, 85%, 90%, 91%, 92%, 93%, 94%, 95%, 96%, 97%, 98%, 99% or more identity to the amino acid sequence shown in SEQ ID NO: 35, or an amino acid sequence having one or more conserved amino acid mutations in comparison to the amino acid sequence of SEQ ID NO: 35,
(12) the heavy chain variable region has the amino acid sequence shown in SEQ ID NO: 59, or a sequence having at least 80%, 85%, 90%, 91%, 92%, 93%, 94%, 95%, 96%, 97%, 98%, 99% or more identity to the amino acid sequence shown in SEQ ID NO: 59, or an amino acid sequence having one or more conserved amino acid mutations in comparison to the amino acid sequence of SEQ ID NO: 59, and
the light chain variable region has the amino acid sequence shown in SEQ ID NO: 60, or a sequence having at least 80%, 85%, 90%, 91%, 92%, 93%, 94%, 95%, 96%, 97%, 98%, 99% or more identity to the amino acid sequence shown in SEQ ID NO: 60, or an amino acid sequence having one or more conserved amino acid mutations in comparison to the amino acid sequence of SEQ ID NO: 60,
(13) the heavy chain variable region has the amino acid sequence shown in SEQ ID NO: 62, or a sequence having at least 80%, 85%, 90%, 91%, 92%, 93%, 94%, 95%, 96%, 97%, 98%, 99% or more identity to the amino acid sequence shown in SEQ ID NO: 62, or an amino acid sequence having one or more conserved amino acid mutations in comparison to the amino acid sequence of SEQ ID NO: 62, and
the light chain variable region has the amino acid sequence shown in SEQ ID NO: 63, or a sequence having at least 80%, 85%, 90%, 91%, 92%, 93%, 94%, 95%, 96%, 97%, 98%, 99% or more identity to the amino acid sequence shown in SEQ ID NO: 63, or an amino acid sequence having one or more conserved amino acid mutations in comparison to the amino acid sequence of SEQ ID NO: 63,
(14) the heavy chain variable region has the amino acid sequence shown in SEQ ID NO: 65, or a sequence having at least 80%, 85%, 90%, 91%, 92%, 93%, 94%, 95%, 96%, 97%, 98%, 99% or more identity to the amino acid sequence shown in SEQ ID NO: 65, or an amino acid sequence having one or more conserved amino acid mutations in comparison to the amino acid sequence of SEQ ID NO: 65, and
the light chain variable region has the amino acid sequence shown in SEQ ID NO: 66, or a sequence having at least 80%, 85%, 90%, 91%, 92%, 93%, 94%, 95%, 96%, 97%, 98%, 99% or more identity to the amino acid sequence shown in SEQ ID NO: 66, or an amino acid sequence having one or more conserved amino acid mutations in comparison to the amino acid sequence of SEQ ID NO: 66,
(15) the heavy chain variable region has the amino acid sequence shown in SEQ ID NO: 37, or a sequence having at least 80%, 85%, 90%, 91%, 92%, 93%, 94%, 95%, 96%, 97%, 98%, 99% or more identity to the amino acid sequence shown in SEQ ID NO: 37, or an amino acid sequence having one or more conserved amino acid mutations in comparison to the amino acid sequence of SEQ ID NO: 37, and
the light chain variable region has the amino acid sequence shown in SEQ ID NO: 38, or a sequence having at least 80%, 85%, 90%, 91%, 92%, 93%, 94%, 95%, 96%, 97%, 98%, 99% or more identity to the amino acid sequence shown in SEQ ID NO: 38, or an amino acid sequence having one or more conserved amino acid mutations in comparison to the amino acid sequence of SEQ ID NO: 38,
(16) the heavy chain variable region has the amino acid sequence shown in SEQ ID NO: 39, or a sequence having at least 80%, 85%, 90%, 91%, 92%, 93%, 94%, 95%, 96%, 97%, 98%, 99% or more identity to the amino acid sequence shown in SEQ ID NO: 39, or an amino acid sequence having one or more conserved amino acid mutations in comparison to the amino acid sequence of SEQ ID NO: 39, and
the light chain variable region has the amino acid sequence shown in SEQ ID NO: 40, or a sequence having at least 80%, 85%, 90%, 91%, 92%, 93%, 94%, 95%, 96%, 97%, 98%, 99% or more identity to the amino acid sequence shown in SEQ ID NO: 40, or an amino acid sequence having one or more conserved amino acid mutations in comparison to the amino acid sequence of SEQ ID NO: 40,
(17) the heavy chain variable region has the amino acid sequence shown in SEQ ID NO: 41, or a sequence having at least 80%, 85%, 90%, 91%, 92%, 93%, 94%, 95%, 96%, 97%, 98%, 99% or more identity to the amino acid sequence shown in SEQ ID NO: 41, or an amino acid sequence having one or more conserved amino acid mutations in comparison to the amino acid sequence of SEQ ID NO: 41, and
the light chain variable region has the amino acid sequence shown in SEQ ID NO: 42, or a sequence having at least 80%, 85%, 90%, 91%, 92%, 93%, 94%, 95%, 96%, 97%, 98%, 99% or more identity to the amino acid sequence shown in SEQ ID NO: 42, or an amino acid sequence having one or more conserved amino acid mutations in comparison to the amino acid sequence of SEQ ID NO: 42,
(18) the heavy chain variable region has the amino acid sequence shown in SEQ ID NO: 43, or a sequence having at least 80%, 85%, 90%, 91%, 92%, 93%, 94%, 95%, 96%, 97%, 98%, 99% or more identity to the amino acid sequence shown in SEQ ID NO: 43, or an amino acid sequence having one or more conserved amino acid mutations in comparison to the amino acid sequence of SEQ ID NO: 43, and
the light chain variable region has the amino acid sequence shown in SEQ ID NO: 44, or a sequence having at least 80%, 85%, 90%, 91%, 92%, 93%, 94%, 95%, 96%, 97%, 98%, 99% or more identity to the amino acid sequence shown in SEQ ID NO: 44, or an amino acid sequence having one or more conserved amino acid mutations in comparison to the amino acid sequence of SEQ ID NO: 44,
(19) the heavy chain variable region has the amino acid sequence shown in SEQ ID NO: 46, or a sequence having at least 80%, 85%, 90%, 91%, 92%, 93%, 94%, 95%, 96%, 97%, 98%, 99% or more identity to the amino acid sequence shown in SEQ ID NO: 46, or an amino acid sequence having one or more conserved amino acid mutations in comparison to the amino acid sequence of SEQ ID NO: 46, and
the light chain variable region has the amino acid sequence shown in SEQ ID NO: 47, or a sequence having at least 80%, 85%, 90%, 91%, 92%, 93%, 94%, 95%, 96%, 97%, 98%, 99% or more identity to the amino acid sequence shown in SEQ ID NO: 47, or an amino acid sequence having one or more conserved amino acid mutations in comparison to the amino acid sequence of SEQ ID NO: 47, or
(20) the heavy chain variable region has the amino acid sequence shown in SEQ ID NO: 67, or a sequence having at least 80%, 85%, 90%, 91%, 92%, 93%, 94%, 95%, 96%, 97%, 98%, 99% or more identity to the amino acid sequence shown in SEQ ID NO: 67, or an amino acid sequence having one or more conserved amino acid mutations in comparison to the amino acid sequence of SEQ ID NO: 67, and
the light chain variable region has the amino acid sequence shown in SEQ ID NO: 68, or a sequence having at least 80%, 85%, 90%, 91%, 92%, 93%, 94%, 95%, 96%, 97%, 98%, 99% or more identity to the amino acid sequence shown in SEQ ID NO: 68, or an amino acid sequence having one or more (preferably 1, 2, 3, 4, 5, 6, 7, 8, 9, 10) conserved amino acid mutations in comparison to the amino acid sequence of SEQ ID NO: 68.

3. The antibody or antigen-binding fragment thereof of claim 1, which binds to TNFR2 of Treg and can kill Treg, with a kill rate of at least 10%, 20%, 30%, 40%, 80%, 85%, 90%, 91%, 92%, 93%, 94%, 95%, 96%, 97%, 98%, 99%, 96%, 97%, 98%, 99% or 100%.

4. The antibody or antigen-binding fragment thereof of claim 1, which binds to TNFR2 of Teff and promotes the proliferation and differentiation of Teff cells.

5. The antibody or antigen-binding fragment thereof of claim 1, which binds to TNFR2 of Teff and promotes Teff bioactivity.

6. The antibody or antigen-binding fragment thereof of claim 1, which has a dissociation constant KD of less than 10 nM with TNFR2.

7. The antibody or antigen-binding fragment thereof of claim 1, which has a dissociation constant KD of less than 1 nM with TNFR2.

8. (canceled)

9. (canceled)

10. (canceled)

11. (canceled)

12. (canceled)

13. (canceled)

14. (canceled)

15. (canceled)

16. (canceled)

17. The antibody or antigen-binding fragment thereof of claim 1, wherein the antibody or antigen-binding fragment thereof comprises murine, human-mouse chimeric, humanized or fully human.

18. The antibody or antigen-binding fragment thereof of claim 1, wherein the antibody or antigen-binding fragment thereof is selected from the group consisting of: monoclonal antibody or antigen-binding fragment thereof, polyclonal antibody or antigen-binding fragment thereof, humanized antibody or antigen-binding fragment thereof, privatized antibody or antigen-binding fragment thereof, bispecific antibody or antigen-binding fragment thereof, multispecific antibody or antigen-binding fragment thereof, dual variable immunoglobulin structural domain, monovalent antibody or antigen-binding fragment thereof, chimeric antibody or antigen-binding fragment thereof, single-chain Fv molecule (scFv), bispecific antibody, trispecific antibody, nanobody, antibody-like protein scaffolds, structural domain antibody, Fv fragment, Fab fragment, F(ab′)2 molecule, and tandem scFv.

19. The antibody or antigen-binding fragment thereof of claim 1, wherein the antibody is a monoclonal antibody.

20. Use of the antibody or antigen-binding fragment thereof of claim 1 in the preparation of a drug comprising the antibody or antigen-binding fragment thereof and a chemotherapy for the treatment of a tumor in a human patient, wherein the antibody and the chemotherapy are formulated to provide a therapeutic effect that is greater than the sum of the respective effects of the reagents.

21. An antibody-drug conjugate, wherein the antibody or antigen-binding fragment thereof of claim 1 is coupled to a therapeutic agent via a linker.

22. The antibody-drug conjugate of claim 21, wherein the therapeutic agent is a cytotoxic agent.

23. A pharmaceutical composition comprising the antibody or antigen-binding fragment thereof of claim 1 and a pharmaceutically acceptable carrier, diluent or excipient.

24. The pharmaceutical composition of claim 23, wherein the composition further comprises an additional therapeutic agent.

25. The pharmaceutical composition of claim 24, wherein the additional therapeutic agent is an immunotherapeutic agent.

26. Use of the antibody or antigen-binding fragment thereof of claim 1 in the following (a), (b), (c), (d) or (e):
(a) preparation of drugs that induce apoptosis in Treg cells;
(b) preparation of drugs that target tumor cell killing;
(c) preparation of drugs that block the TNF-TNFR2 tumor signaling pathway;
(d) preparation of drugs that promote the proliferation, differentiation and/or biological activity of Teff cells; or
(e) preparation of drugs for the treatment of autoimmune diseases.

27. The use of claim 26, wherein the tumor is selected from the group consisting of: ovarian cancer, melanoma, prostate cancer, intestinal cancer, gastric cancer, oesophageal cancer, breast cancer, lung cancer, renal cancer, pancreatic cancer, uterine cancer, liver cancer, bladder cancer, cervical cancer, oral cavity cancer, brain cancer, testicular cancer, skin cancer, colorectal cancer, malignant glioma, and thyroid cancer.
